(19) United States
(12) Patent Application Publication
(10) Pub. No.: US 2015/0329493 A1
(43) Pub. Date: Nov. 19, 2015

(54) OMEGA-AMINO ACID DERIVATIVES OF BENZENE, PYRIDINE, AND PYRIDAZINE COMPOUNDS

(71) Applicant: Steven E. Hall, Chapel Hill, NC (US)

(72) Inventor: Steven E. Hall, Chapel Hill, NC (US)

(21) Appl. No.: 14/716,002

(22) Filed: May 19, 2015

Related U.S. Application Data
(60) Provisional application No. 62/000,231, filed on May 19, 2014.
(60) Provisional application No. 62/000,391, filed on May 19, 2014.

(51) Int. Cl. C07D 231/56; C07D 401/12; C07D 405/12; C07D 209/08; C07D 403/12

(52) U.S. Cl. CPC C07D 231/56; C07D 209/08; C07D 403/12; C07D 405/12; C07D 401/12

(57) ABSTRACT

Disclosed are compounds and pharmaceutically acceptable salts of Formula I

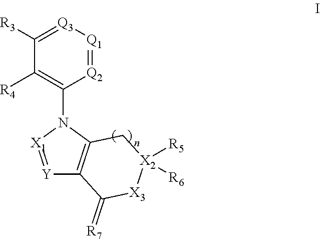

wherein R₁, R₂, R₃, R₄, R₅, R₆, R₇, n, Q₁, Q₂, Q₃, Y, and X₁-X₄ are as defined herein. Compounds of Formula I are useful in the treatment of diseases and/or conditions related to cell proliferation, such as cancer, inflammation, arthritis, angiogenesis, or the like. Also disclosed are pharmaceutical compositions comprising compounds of the invention and methods of treating the aforementioned conditions using such compounds.

### CROSS-REFERENCE TO RELATED APPLICATIONS

This application claims priority to U.S. Provisional Application No. 62/000,231, filed May 19, 2014, and to U.S. Provisional Application No. 62/000,391, filed May 19, 2014, the disclosure of each of which is hereby incorporated by reference in its entirety.

### BACKGROUND OF THE INVENTION

1. Field of the Invention

The invention relates to benzene, pyridine, and pyridazine derivatives and more specifically to such compounds that are useful in the treatment and/or prevention of diseases and/or conditions related to cell proliferation, such as cancer, inflammation and inflammation-associated disorders, and conditions associated with angiogenesis. Compounds of the invention are also useful in the treatment and/or prevention of infectious diseasaes, in particular, fungal infections.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, and the like.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have antiproliferative and antitumor activities.

HSP-90 is a molecular chaperone that guides the normal folding, intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 will slow those process thus has potential therapeutic use (Whitesell L, Lindquist, S L, Nature Rev. Cancer, 2005, 10, 761-72).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP-90, thereby destabilizing substrates that normally interact with HSP-90 (Stebbins, C. et al. Cell 1997, 89, 239-250). This pocket is highly conserved and has weak homology to the ATP-binding siteof DNA gyrase (Stebbins, C. et al., supra ; Grenert, J. P. et al. J. Biol. Chem. 1997,272,23843-50).

In vitro and in vivo studies have demonstrated that occupancy of this N-terminal pocket by ansamycins and other HSP-90 inhibitors alters HSP-90 function and inhibits protein folding. At high concentrations, ansamycins and other HSP-90 inhibitors have been shown to prevent binding of protein substrates to HSP-90 (Scheibel , T. H. et al. Proc. Natl. Acad. Sci. USA 1999, 96, 1297-302; Schulte, T. W. et al.J. Biol. Chem. 1995,270,24585-8; Whitesell, L. , et al. Proc. Natl.Acad. Sci. USA 1994, 91, 8324-8328). Ansamycins have also been demonstrated to inhibit the ATP-dependent release of chaperone-associated protein substrates (Schneider, C. L. et al. Proc.Natl. Acad. Sci., USA 1996, 93, 14536-41; Sepp-Lorenzinoet al. J. Biol Chem. 1995,270,16580-16587). In either event, the substrates are degraded by a ubiquitin-dependent process in the proteasome (Schneider, C. L., supra; Sepp-Lorenzino, L. , et al. J. Biol.Claim. 1995,270, 16580-16587; Whitesell, L. et al. Proc. Natl.Acad. Sci. USA 1994, 91, 8324-8328). HSP-90 substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239, 655-9 Schulte, T. W., et al., J. Biol. Chem. 1995,270, 24585-8), nuclear steroid receptors(Segnitz, B.; U. Gehring J. Biol. Chem. 1997, 272, 18694-18701 ; Smith, D. F. et al. Mol. Cell Biol. 1995,15, 6804-12), v-Src (Whitesell, L. , et al. Proc. Natl.Acad. Sci. USA 1994, 91, 8324-8328) and certain transmembrane tyrosine kinases (Sepp-Lorenzino, L. et al. J. Biol.Chez. 1995,270, 16580-16587) such as EGF receptor (EGFR) and HER2/Neu(Hartmann, F. , et al. Int. J. Cancer 1997,70, 221-9; Miller, P. et al.CancerRes. 1994,54, 2724-2730; Mimnaugh, E. G. , et al.J.Biol. Clzem. 1996,271, 22796-801 ; Schnur, R. et al. J. Med.Chenu. 1995, 38,3806-3812), CDK4, and mutant p53. Erlichman et al. Proc. AACR 2001, 42, abstract 4474. The ansamycin-induced loss of these proteins leads to the selective disruption of certain regulatory pathways and results in growth arrest at specific phases of the cell cycle (Muise-Heimericks, R. C. et al. J. Biol. Chez. 1998, 273, 29864-72), and apoptosis, and/or differentiation of cells so treated (Vasilevskaya, A. et al. CancerRes., 1999,59, 3935-40). Inhibitors of HSP-90 thus hold great promise for the treatment and/or prevention of many types of cancers and proliferative disorders, and also hold promise as traditional antibiotics.

Inhibition of HSP-90 is also known to result in up regulation of the expression of the chaperone HSP70. HSP70 up regulation is considered to be of therapeutic benefit for treatment of a wide range of neurodegenerative diseases including, but not limited to: Alzheimer's disease; Parkinson's disease; Dementia with Lewy bodies; Amyotropic lateral scleriosis (ALS); Polyglutamine disease; Huntington's disease; Spinal and bulbar muscular atrophy (SBMA); and Spinocerebellar ataxics (SCA1-3,7). Therefore, the compounds described in the invention are of potential therapeutic use for treatment of such neurodegenerative diseases (Muchowski, P. J., Wacker J. L., Nat. Rev. Neurosci. 2005, 6, 11-22.; Shen H. Y., et al. J. Biol. Chem. 2005, 280, 39962-9).

Therefore, there is a continuing need in the art for new methods of treating cancer, inflammation and inflammation-associated disorders, and conditions or diseases related to uncontrolled angiogenesis.

### SUMMARY OF THE INVENTION

In a broad aspect, the invention encompasses the compounds of formula I shown below, pharmaceutical compositions containing those compounds and methods employing such compounds or compositions in the treatment of diseases and/or conditions related to cell proliferation, such as cancer, inflammation, arthritis, angiogenesis, or the like.

The invention provides compounds of formula I,

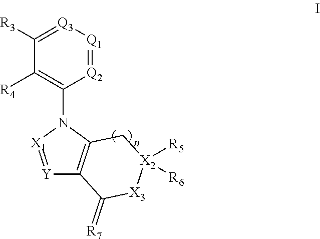

and pharmaceutically acceptable salts thereof, wherein
R₃ and R₄ are independently

(a) H,

(b) halo, or

(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
R₂₂ is
   (i) heteroaryl,
   (ii) aryl,
   (iii) saturated or unsaturated C₃-C₁₀ cycloalkyl, or
   (iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S-(C₁-C₆)alkyl, —SO₂-(C₁-C₆)alkyl, —SO₂-aryl, —SO-(C₁-C₆)alkyl, —SO-aryl, —SO₂NH₂, —SO₂NH-(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;

wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, —SO₂NH-(C₁-C₆)alkyl, —SO₂NH-aryl, —SO₂-aryl, —SO-(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —O C₁-C₁₀ alkyl-Z, or R₂₃, wherein
Z is OR₀ or —N(R₃₀)₂, wherein
   each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
   Rₒ is —H, -C₁-C₁₀ alkyl, -C₂-C₁₀ alkenyl, -C₂-C₁₀ alkynyl, aryl, heteroaryl, or -C₁-C₆ acyl;
R₂₃ is
   (1) heteroaryl,
   (2) aryl,
   (3) saturated or unsaturated C₅-C₁₀ cycloalkyl, or
   (4) saturated or unsaturated C₅-C₁₀ heterocycloalkyl, and the R₂₃ groups are optionally substituted with at least one group which independently is hydroxy, oxo, halo, amino, cyano, nitro, —SH, —S-(C₁-C₆)alkyl, —SO₂-(C₁-C₆)alkyl, —SO₂-aryl, —SO-(C₁-C₆)alkyl, —SO-aryl, —SO₂NH₂, —SO₂NH-(C₁-C₆)alkyl, —SO₂NH-aryl, (C₁-C₆)alkoxy, or mono- or di-(C₁-C₁₀)alkylamino; and

wherein at least one of R₃ and R₄ is substituted with a group R₅₀ where R₅₀ is:

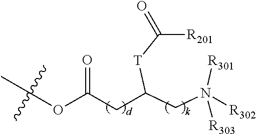

wherein
d and k are integers independently selected from 1 and 2;
R₂₀₁ is (C₁-C₆)alkyl where the alkyl is optionally substituted with (C₃-C₇)cycloalkyl, (C₂-C₆) alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;

R₇ is O, S, NH, N—OH, N—NH₂, N—NHR₂₂, N—NH-(C₁-C₆ alkyl), N—O-(C₀-C₆)alkyl-R₂₂, N-(C₁-C₆ alkenoxy);or N-(C₁-C₆ alkoxy optionally substituted with carboxy);

Y is N or CR_{C}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
   each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, cyano, nitro, halo(C₁-C₆)alkyl, halo(C₁-C₆)alkoxy, carboxamide, heterocycloalkyl, aryl, or heteroaryl, wherein
      the aryl and heteroaryl groups are optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, or carboxamide;
   R_{C′} is —C₁-C₆ alkyl, —OR_{C″}, or —N(R_{CN})_{2,} wherein
      R_{C″} is —H, C₁-C₁₀ alkyl, C₁-C₁₀ haloalkyl, C₃-C₇ cycloalkyl, heterocycloalkyl, aryl, or heteroaryl;
      each R_{CN} is independently —H, -C₁-C₁₀ alkyl, -C₁-C₁₀ -aloalkyl, -C₃-C₇ cycloalkyl, -heterocycloalkyl, -C₁-C₆ acyl, -aryl, or -heteroaryl, wherein
      each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;

X₁ is N or CR_{C};

Q₁, Q_{2,} and Q₃ are independently N or CR_{Q} , wherein one and only one of Q₁, Q₂, and

Q3 is C-R₂₁, and wherein
each R_{Q} is independently hydrogen, halogen, —N(R_{cN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula

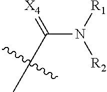

wherein
   R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
      each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN}); and
X₄ is O, S, NH, NOH, N—NH₂, N—NHaryl, N—NH-(C₁-C₆ alkyl), or N-(C₁-C₆ alkoxy);
   X₂ and X₃ are independently C, O, N, or S(O)ₚ wherein

p is 0, 1, or 2; and

n is 0, 1, 2, 3, or 4;
provided that when

(i) X₂ is C, then

R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide,
wherein any two adjacent substituted aryl positions, together with the carbon atoms to which they are attached, form an unsaturated cycloalkyl or heterocycloalkyl; or

R₅ and R₆ together with the carbon to which they are attached form a 3-8 membered ring;

(ii) X₂ is N, thenR₆ is absent and R₅ is H or C₁-C₆ alkyl;

(iii) X₃ is C, then it is substituted with two groups that are independently H, C₁-C₆ alkyl, or mono- or di-(C₁-C₆)alkylamino(C₁-C₆)alkyl; and

(iv) X₂ is 0 or S(O)ₚ, then R₆ and R₅ are absent.

The invention also includes intermediates that are useful in making the compounds of the invention.

The invention also provides pharmaceutical compositions comprising a compound or pharmaceutically acceptable salt of Formula I and at least one pharmaceutically acceptable carrier, solvent, adjuvant or diluent.

The invention further provides methods of treating disease such as cancer, inflammation, arthritis, angiogenesis, and infection in a patient in need of such treatment, comprising administering to the patient a compound or pharmaceutically acceptable salt of Formula I, or a pharmaceutical composition comprising a compound or salt of Formula I.

The invention also provides the use of a compound or salt according to Formula I for the manufacture of a medicament for use in treating cancer, inflammation, arthritis, angiogenesis, or infection.

The invention also provides methods of preparing the compounds of the invention and the intermediates used in those methods.

The invention also provides methods of treating a disease or condition related to cell proliferation comprising administering a therapeutically effective amount of a compound or salt of Formula I to a patient in need of such treatment.

The invention also provides methods of treating a disease or condition related to cell proliferation comprising administering a therapeutically effective amount of a compound or salt of Formula I to a patient in need of such treatment, where the disease of condition is cancer, inflammation, or arthritis.

The invention further provides methods of treating a subject suffering from a disease or disorder of proteins that are either client proteins for HSP-90 or indirectly affect its client proteins, comprising administering to a subject in need of such treatment a therapeutically effective amount of a compound or salt of Formula I.

The invention further provides a compound or pharmaceutical composition thereof in a kit with instructions for using he compound or composition.

The invention further provides compounds that may be administered alone or in combination with other drugs or therapies known to be effective to treat the disease to enhance overall effectiveness of therapy.

### DETAILED DESCRIPTION OF THE INVENTION

In Formula I, R₃ and R₄ are, as noted above, independently (a) hydrogen, (b) halo, or (c) an alkyl group having from 1-15 carbon atoms. All, but no more than about six, of the carbon atoms in the alkyl group may be replaced independently by the various groups listed above in connection with Formula I.

Thus, when the alkyl group is methyl, i.e., a one carbon atom alkyl group, replacement of that carbon atom with, for example, nitrogen or sulfur, the resulting group will not be an alkyl group but instead will be an amino or thio group, respectively. Similarly, when the carbon atom being replaced terminates the alkyl group, the terminal group will become another moiety such as pyrimidinyl, amino, phenyl, or hydroxy.

Replacement of a carbon atom with a group such as, for example, oxygen, nitrogen, or sulfur will require appropriate adjustment of the number of hydrogens or other atoms required to satisfy the replacing atom's valency. Thus, when the replacement is N or O, the number of groups attached to the atom being replaced will be reduced by one or two to satisfy the valency of the nitrogen or oxygen respectively. Similar considerations will be readily apparent to those skilled in the art with respect to replacement by ethenyl and ethynyl.

Thus, replacement as permitted herein results in the term “C₁-C₁₅ alkyl” as defined in connection with Formula I encompassing groups such as, but not limited to:
amino, hydroxy, phenyl, benzyl, propylaminoethoxy, butoxyethylamino, pyrid-2-ylpropyl, diethylaminomethyl, pentylsulfonyl, methylsulfonamidoethyl, 3-[4-(butylpyrimidin-2-yl)ethyl]phenyl, butoxy, dimethylamino, 4-(2-(benzylamino)ethyl)pyridyl, but-2-enylamino, 4-(1-(methylamino)pent-3-en-2-ylthio)phenyl, 2-(N-methyl-hexanamido)ethoxy)methyl, and 4-(((3-methoxy-4-(4-methyl-1 H-imidazol-2-yl)but-1-enyl)(methyl)amino)-methyl)phenyl.

Preferred compounds of Formula I include those where X₁ is carbon optionally substituted with C₁-C₆ alkyl, more preferably C₁-C₃ alkyl. Other preferred compounds of Formula I are those where X₁ is carbon optionally substituted with C₁-C₆ alkyl and Y is CR_{C} wherein R_{C} is —H, C₁-C₆ alkyl, C₁-C₃ haloalkyl, C₃-C₇ cycloalkyl, or C₃-C₇ cycloalkyl(C₁-C₆)alkyl. More preferably, in compounds of Formula I, X₁ is carbon optionally substituted with C₁-C₂ alkyl and Y is CR_{C} wherein R_{C} is —H, C₁-C₄ alkyl, C₁-C₃ haloalkyl, cyclopropyl, or cyclopropyl(C₁-C₂)alkyl.

Still more preferred compounds of Formula I are those where X₁ is CH. Other more preferred compounds of Formula I are those where X₁ is CH and Y is CR_{C} wherein R_{C} is —H, C₁-C₃ alkyl, C₁-C₃ haloalkyl, C₃-C₅ cycloalkyl, or C₃-C₅ cycloalkyl(C₁-C₂)alkyl. Even more preferred compounds of Formula I are those where X₁ is CH and Y is CR_{C} wherein R_{C} is —H, methyl, ethyl, trifluoromethyl, cyclopropyl, or cyclopropylmethyl. Particularly preferred compounds of Formula I are those where X₁ is CH and Y is CR_{C} wherein R_{C} is methyl, ethyl, or cyclopropyl. Other particularly preferred compounds of Formula I are those where X₁ is CH and Y is CR_{C} wherein R_{C} is trifluoromethyl. Other particularly preferred compounds of Formula I are those where X₁ is CH and Y is CR_{C} wherein R_{C} is methyl. Other particularly preferred compounds of Formula I are those where X₁ is CH and Y is CR_{C} wherein R_{C} is ethyl. Other particularly preferred compounds of Formula I are those where X₁ is CH and Y is CR_{C} wherein R_{C} is cyclopropyl.

Still more preferred compounds of Formula I are those where X₁ is N. Other more preferred compounds of Formula I are those where X₁ is N and Y is CR_{C} wherein R_{C} is —H, C₁-C₃ alkyl, C₁-C₃ haloalkyl, C₃-C₅ cycloalkyl, or C₃-C₅ cycloalkyl(C₁-C₂)alkyl. Even more preferred compounds of Formula I are those where X₁ is N and Y is CR_{C} wherein R_{C} is —H, methyl, ethyl, trifluoromethyl, cyclopropyl, or cyclopropylmethyl. Particularly preferred compounds of Formula I are those where X₁ is N and Y is CR_{C} wherein R_{C} is methyl, ethyl, or cyclopropyl. Other particularly preferred compounds of Formula I are those where X₁ is N and Y is CR_{C} wherein R_{C} is trifluoromethyl. Other particularly preferred compounds of Formula I are those where X₁ is N and Y is CR_{C} wherein R_{C} is methyl. Other particularly preferred compounds of Formula I are those where X₁ is N and Y is CR_{C} wherein R_{C} is ethyl. Other particularly preferred compounds of Formula I are those where X₁ is N and Y is CR_{C} wherein R_{C} is cyclopropyl.

Other preferred compounds of Formula I are those where n is 1, X₂ is CH₂, X₃ is carbon, and R₅ and R₆ are both methyl.

Other preferred compounds of Formula I are those where Q3 is CR₂₁, wherein

R₂₁ is a group of the formula,

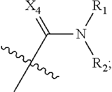

R₇ is O; and

Y is CR_{C}, wherein R_{C} is hydrogen, C₁-C₃ alkyl, C₃-C₅ cycloalkyl, trifluoromethyl, or C₃-C₅ cycloalkyl(C₁-C₂)alkyl. Such compounds are compounds of Formula II herein.

Other preferred compounds of Formula I are those where

Q₃ is CR₂₁, wherein

R₂₁ is a group of the formula,

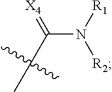

and
X₃ is C substituted with R₉ₐ and R_{9b}, wherein R₉ₐ and R_{9b} are independently H or C₁-C₆ alkyl. Such compounds are hereinafter compounds of Formula III.

Other preferred compounds of Formula I are those where

Q3 is CR₂₁, wherein

R₂₁ is a group of the formula,

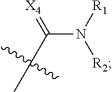

and

Q₁ and Q₂ are independently C substituted with R₁₀ₐ and R_{10b} respectively, wherein R₁₀ₐ and R_{10b} are independently H or C₁-C₆ alkyl. Such compounds are hereinafter compounds of Formula IV.

Other preferred compounds of Formula I are those where

Q3 is CR₂₁, wherein

R₂₁ is a group of the formula,

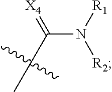

and

X₁ is C substituted with R₁₁ where R₁₁ hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
R_{C′} is -C₁-C₆ alkyl, —OR_{C″}, or —N(R_{CN})_{2,} wherein
   R_{C″} is —H, C₁-C₁₀ alkyl, C₁-C₁₀ haloalkyl, C₃-C₇ cycloalkyl, heterocycloalkyl, aryl, or heteroaryl;
      each R_{CN} is independently —H, -C₁-C₁₀ alkyl, -C₁-C₁₀ -haloalkyl, -C₃-C₇ cycloalkyl, -heterocycloalkyl, -C₁-C₆ acyl, -aryl, or -heteroaryl. Such compounds are hereinafter compounds of Formula V.

Preferred compounds of Formula V are those where R₁₁ is hydrogen, halogen, C₁-C₁₀ alkyl, C₁-C₁₀ haloalkyl, C₃-C₇ cycloalkyl, C₃-C₇ cycloalkyl(C₁-C₁₀)alkyl, aryl, or heteroaryl.

More preferred compounds of Formula V are those where R₁₁ is H or C₁-C₆ alkyl.

Other preferred compounds of Formula I are those where

Q₃ is CR₂₁, wherein

R₂₁ is a group of the formula,

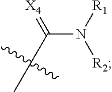

and

X₁ is N. Such compounds are hereinafter compounds of Formula Va.

Other preferred compounds of Formula I are those where

Q₃ is CR₂₁, wherein

R₂₁ is a group of the formula,

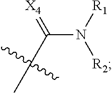

and

X₂ is C substituted with R₅ and R₆, wherein R₅ and R₆ are independently H or C₁-C₄ alkyl.
Such compounds are hereinafter compounds of Formula VI.

More preferred compounds of the invention are those of Formula I wherein Q₃ is CR₂₁, wherein

More preferred compounds of the invention are those of Formula I wherein

R₁ and R₂ are independently H or C₁-C₄ alkyl;

Q₁ and Q₂ are both CH;

X₂ is C substituted with two independently selected C₁-C₄ alkyl groups; and

n is 1.

Other preferred compounds of the invention include those having the formula VII

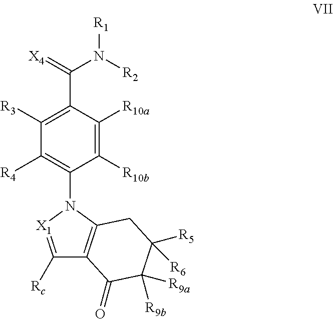

wherein X₁ and R_{C} are as defined in Formula I;
R₅ and R₆ are independently H or C₁-C₄ alkyl;
R₁₁ is H or C₁-C₆ alkyl;
R₁₀ₐ and R_{10b} are independently H or C₁-C₆ alkyl;
R₉ₐ and R_{9b} are independently H or C₁-C₆ alkyl.

Preferred compounds of Formula VII include those where

R₁ and R₂ are independently H or C₁-C₄ alkyl;
R₁₀ₐ and R_{10b} are both H; and
R₅ and R₆ are independently C₁-C₄ alkyl.

Other preferred compounds of Formula VII include those where

### X₁ is N.

Other preferred compounds of Formula VII include those where X₁ is CR_{C}, wherein R_{C} is hydrogen, methyl, ethyl, cyclopropyl, cyclopropylmethyl, fluoromethyl, difluoromethyl, or trifluoromethyl. In a preferred embodiment of this aspect, the R_{c} group derived from X₁ is hydrogen, methyl, or trifluoromethyl, and the R_{c} group derived from Y carries the definition given in connection with Formula I.

Other preferred compounds of Formula I include those of formula VIII,

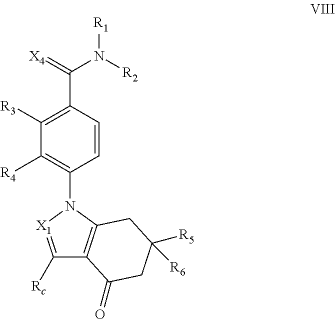

wherein R_{C} is H, C₁-C₆ alkyl, trifluoromethyl, or cyclopropyl; and
R₁-R₆, X₁,and X₄ carry the same definitions as for Formula I.

Preferred compounds of Formula VIII include those where

### X₁ is N.

Preferred compounds of Formula VIII include those where X₁ is CR_{C}, wherein R_{C} is hydrogen, methyl, ethyl, cyclopropyl, cyclopropylmethyl, fluoromethyl, difluoromethyl, or trifluoromethyl. In a preferred embodiment of this aspect, the R_{c} group derived from X₁ is hydrogen, methyl, or trifluoromethyl, and the R_{c} group derived from Y carries the definition given in connection with Formula I.

Other preferred compounds of Formula I are those of Formula IX:

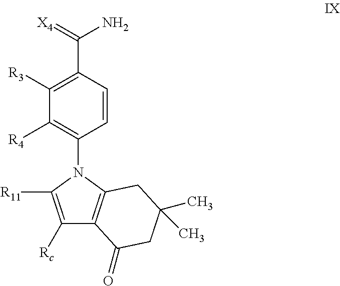

where R₁₁ is hydrogen or methyl, preferably hydrogen;
R_{C} is H, C₁-C₂ alkyl, trifluoromethyl, or cyclopropyl; and
R₃, R₄, and X₄ carry the same definitions as for Formula I. Preferred compounds of Formula IX include those where R_{C} is C₁-C₂ alkyl, trifluoromethyl, or cyclopropyl.

Other preferred compounds of Formula I are those where R₂₁ is cyano, R₇ is O, and Y is CR_{C}, wherein R_{C} is H, methyl, ethyl, trifluoromethyl, or cyclopropyl.

Other preferred compounds of Formula I are those where, R₂₁ is cyano; R₇ is O; and Y is CR_{C}, wherein R_{C} is H, methyl, trifluoromethyl, or cyclopropyl.

Yet other preferred compounds of Formula I are those where R₂₁ is cyano, and X₃ is C substituted with two groups that are independently H or C₁-C₆ alkyl.

More preferred compounds of Formula I are those where R₂₁ is cyano, and Q₁ and Q₂ are independently C substituted with H or C₁-C₆ alkyl.

Yet other preferred compounds of Formula I are those where R₂₁ is cyano, and X₁ is C substituted with H or C₁-C₆ alkyl.

Still other preferred compounds of Formula I are those where R₂₁ is cyano, and X₂ is C substituted with two groups that are independently H or C₁-C₄ alkyl.

Yet other preferred compounds of Formula I include those of Formula X,

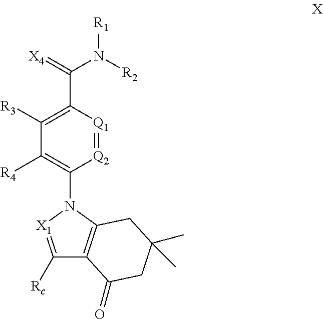

wherein X₁-X₄, Q₁, Q₂, R_{C}, and R₁-R₄ are as defined in Formula I.

Preferred compounds of formula X are those where Q₁ and Q₂ are each independently hydrogen or C₁-C₆ alkyl.

Other preferred compounds of formula X are those where R_{c} is C₁-C₆alkyl, C₃-C₇cycloalkyl, C₁-C₆ haloalkyl, C₃-C₇cycloalkyl(C₁-C₆)alkyl, or heterocycloalkyl.

More preferred compounds of Formula X include those where R_{c} is C₃-C₇cycloalkyl, C₁-C₆ haloalkyl, heterocycloalkyl, or C₃-C₇cycloalkyl(C₁-C₆)alkyl.

Particularly preferred compounds of Formula X include those where R_{c} is C₁-C₃alkyl, C₃-C₅cycloalkyl, C₃-C₅cycloalkyl(C₁-C₃)alkyl, or C₁-C₂ haloalkyl.

Preferred compounds of Formula X are those where X₁ is N. Such compounds are referred to herein as compounds of Formula XI.

Preferred compounds of any of Formulas I-X include those wherein R₃ is substituted with R₅₀, and R₅₀ is

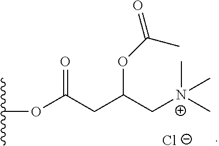 [SMILES: *OC(=O)CC(C[N+](C)(C)C)OC(C)=O ; [Cl-]]

Other preferred compounds of any of Formulas I-X include those wherein
Q₃ is CR₂₁, wherein
   R₂₁ is a group of the formula,

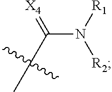

R₇ is O; and
Y is CR_{C}, wherein
   R_{C} is —H, —CH₃, ethyl, cyclopropyl, or —CF₃.
Other preferred compounds of any of Formulas I-X include those wherein
Q₃ is CR₂₁, wherein
   R₂₁ is a group of the formula,

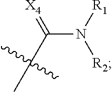

and X₂ is C substituted with two groups that are independently H or C₁-C₆ alkyl.

Other preferred compounds of any of Formulas l-X include those wherein Q₃ is CR₂₁, wherein
R₂₁ is a group of the formula,

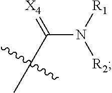

and Q₁ and Q₂ are independently C substituted with H or C₁-C₆ alkyl.

Other preferred compounds of any of Formulas I-X include those wherein X₁ is N.

Other preferred compounds of any of Formulas I-X include those wherein X₁ is CR_{c}.

Other preferred compounds of any of Formulas I-X include those wherein Q₃ is CR₂₁, wherein
R₂₁ is a group of the formula,

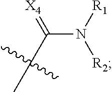

and X₁ is C substituted with H or C₁-C₆ alkyl.

Other preferred compounds of any of Formulas l-X include those wherein Q₃ is CR₂₁, wherein
R₂₁ is a group of the formula,

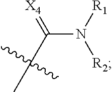

and X₂ is C substituted with two groups that are independently H or C₁-C₄ alkyl.

Other preferred compounds of any of Formulas I-X include those wherein R₂₁ is a group of the formula,

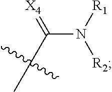

R₃ is —Z₁R_{Z1}, wherein
   Z₁ is —O— or —NH—; and
   R_{Z1} is a saturated or unsaturated C₃-C₁₀ cycloalkyl, each of which is
      (a) substituted with R₅₀, where R₅₀ is

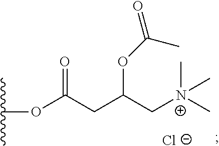 [SMILES: *OC(=O)CC(C[N+](C)(C)C)OC(C)=O ; [Cl-]]

and
(b) optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, —SO₂NH-(C₁-C₆)alkyl, —SO₂NH-aryl, —SO₂-aryl, —SO-(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —O C₁-C₁₀ alkyl-Z, or R₂₃; and
R₄ is H or halogen.

Other preferred compounds of any of Formulas I-X include those wherein R_{Z1} is a saturated C₅-C₇ cycloalkyl.

Other preferred compounds of any of Formulas I-X include those wherein wherein R_{Z1} is a unsaturated C₅-C₇ cycloalkyl.

Other preferred compounds of any of Formulas I-X include those wherein X₁ is N.

Other preferred compounds of any of Formulas I-X include those wherein X₁ is CR_{c}.

Other preferred compounds of any of Formulas I-X include those wherein X₁ is CH.

Other preferred compounds of any of Formulas I-X include those wherein
R₁ and R₂ are independently H or C₁-C₄ alkyl;
Q₁ and Q₂ are both CH;
X₂ is C substituted with two independently selected C₁-C₄ alkyl groups; and
n is 1.

Other preferred compounds of any of Formulas I-X include those of the formula,

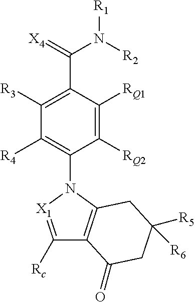

wherein

R_{Q1} is H or halogen; and

R_{Q2} is H or halogen.

Other preferred compounds of any of Formulas I-X include those wherein
R₃ is cyclohexyl which is
(a) substituted with R₅₀, where R₅₀ is

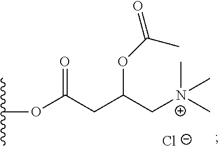 [SMILES: *OC(=O)CC(C[N+](C)(C)C)OC(C)=O ; [Cl-]]

and
(b) optionally substituted at any available position with C₁-C₁₀ alkyl, C₁⁻C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, —SO₂NH-(C₁-C₆)alkyl, —SO₂NH-aryl, —SO₂-aryl, —SO-(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₃; and
R₄ is H or fluoro.

In another aspect, the invention encompasses a method of treating cancer comprising administering to a patient in need thereof, a pharmaceutically acceptable amount of a compound or salt of Formula I or a pharmaceutical composition comprising a compound or salt of Formula I.

In another aspect, the invention encompasses the use of a therapeutically effective amount of a compound or salt of Formula I for the preparation of a medicament for the treatment of cancer, inflammation, or arthritis in a patient in need of such treatment.

In another aspect, the invention encompasses a package comprising a compound or salt of Formula I in a container with instructions on how to use the compound.

In another aspect, the invention encompasses the use of a therapeutically effective amount of a compound or salt according of Formula I for the preparation of a medicament for the treatment of a disease or condition related to cell proliferation in a patient in need of such treatment.

In another aspect, the invention encompasses the use of a therapeutically effective amount of a compound or salt according of Formula I for the preparation of a medicament for the treatment of a disease or condition related to cell proliferation in a patient in need of such treatment, wherein the disease or condition is cancer, inflammation, or arthritis.

In another aspect, the invention encompasses the use of therapeutically effective amount of a compound or salt of Formula I for the preparation of a medicament for the treatment of a disease or disorder related to the activity of heat shock protein 90, in a subject in need of such.

In another aspect, the invention encompasses the use of therapeutically effective amount of a compound or salt of Formula I, alone or in combination with another therapeutic agent, for the preparation of a medicament for the treatment of a disease or disorder related to the activity of heat shock protein 90 and/or its client protiens, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In a preferred aspect, the invention encompasses methods for the treatment of cancer in a subject in need of such treatment comprising administration of therapeutically effective amount of a compound or salt of Formula I, in combination with at least one other therapeutic agent.

In a more preferred aspect, the invention encompasses methods for treating cancer in a subject in need of such treatment, the methods comprising administration of therapeutically effective amount of a compound or salt of Formula I, in combination with at least one other anti-cancer agent.

In another preferred aspect, the invention encompasses methods for treating cancer, the methods comprising administration, to a subject in need of such treatment, of a therapeutically effective amount of a compound or salt of Formula I, in combination with radiation therapy.

### DEFINITIONS

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designated number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenoxy” refers to an alkenyl group attached to the parent group through an oxygen atom.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl, mono- and di(C₁-C₈alkyl)amino(C₁-C₈)alkyl, C₁-C₈acyl, C₁-C₈acyloxy, C₁-C₈sulfonyl, C₁-C₈thio, C₁-C₈sulfonamido, C₁-C₈aminosulfonyl .

The term “carboxy” as used herein, means a —CO₂H group.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃. A preferred haloalkoxy group is trifluoromethoxy. The term “haloalkyl” refers to an alkyl group substituted with one or more halogen atoms, where each halogen is independently F, Cl, Br or I. Preferred halogens are F and Cl.

Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀ cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E- configurations. Likewise, all tautomeric forms are also intended to be included.

### Pharmaceutical Compositions

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients.

The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the present invention may be administed alone or in combination with at least one additional therapeutic agent or therapy, e.g., radiation therapy, to a patient in need of such treatment. The additional therapeutic agent or therapy may be administed at the same time, separately, or sequentially with respect to the administration of a compound of the invention. Such additional therapeutic agents included, but are not limited to, anti-cancer agents, anti-inflammatory agents, and the like.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

### Methods of Preparation

### General Procedure

Representative synthetic procedures for the preparation of compounds of the invention are outlined below in following schemes. Unless otherwise indicated, X₁, X₂, X₃, n, R₅, R₆, R₇, R_{C}, R₁₁, and Y carry the definitions given in connection with Formula I. R is selected from moieties appropriate to provide a substituted amino group falling within the definition of R₃ in Formula I.

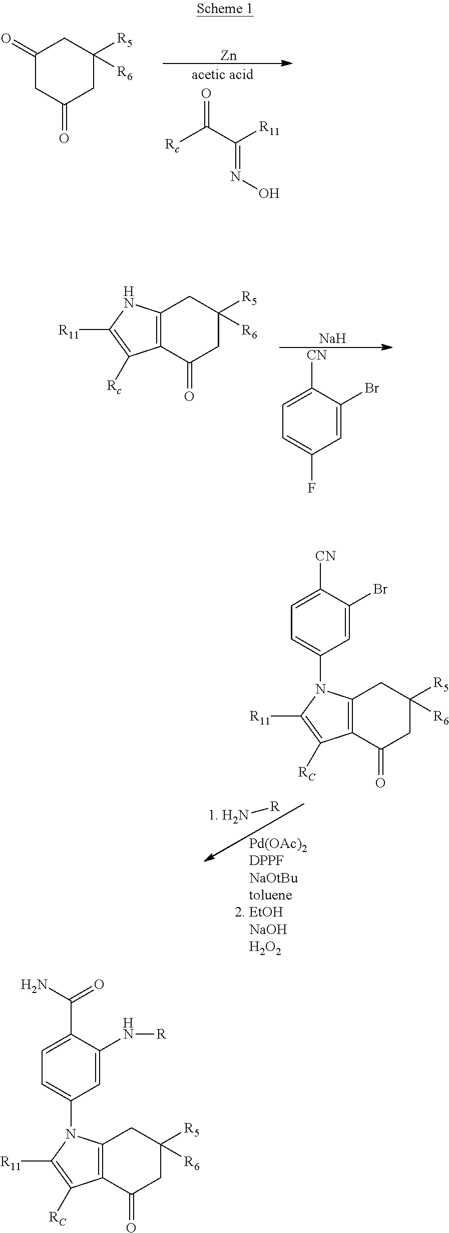

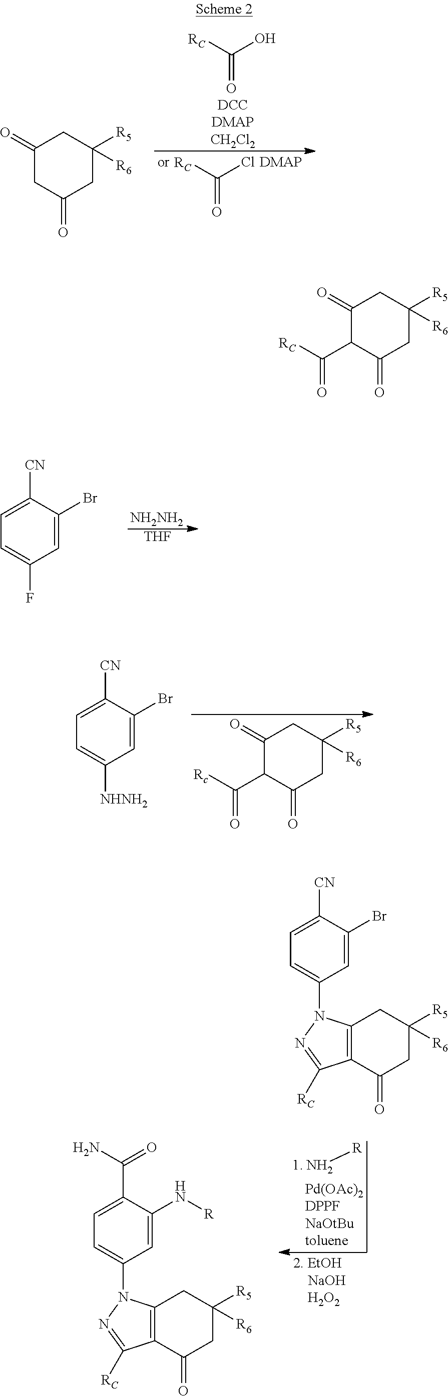

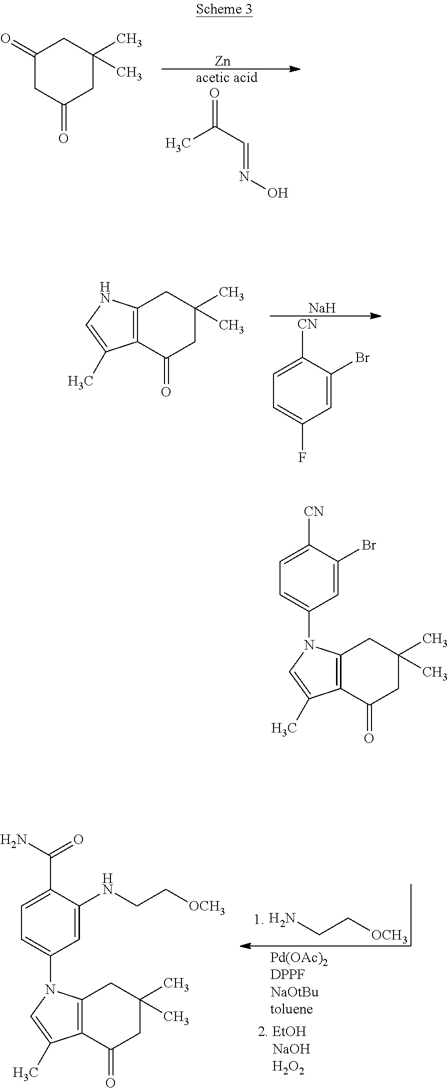

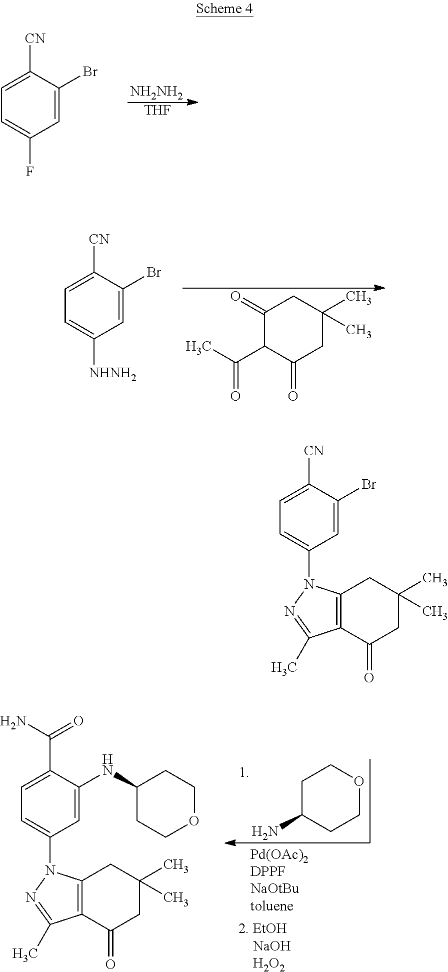

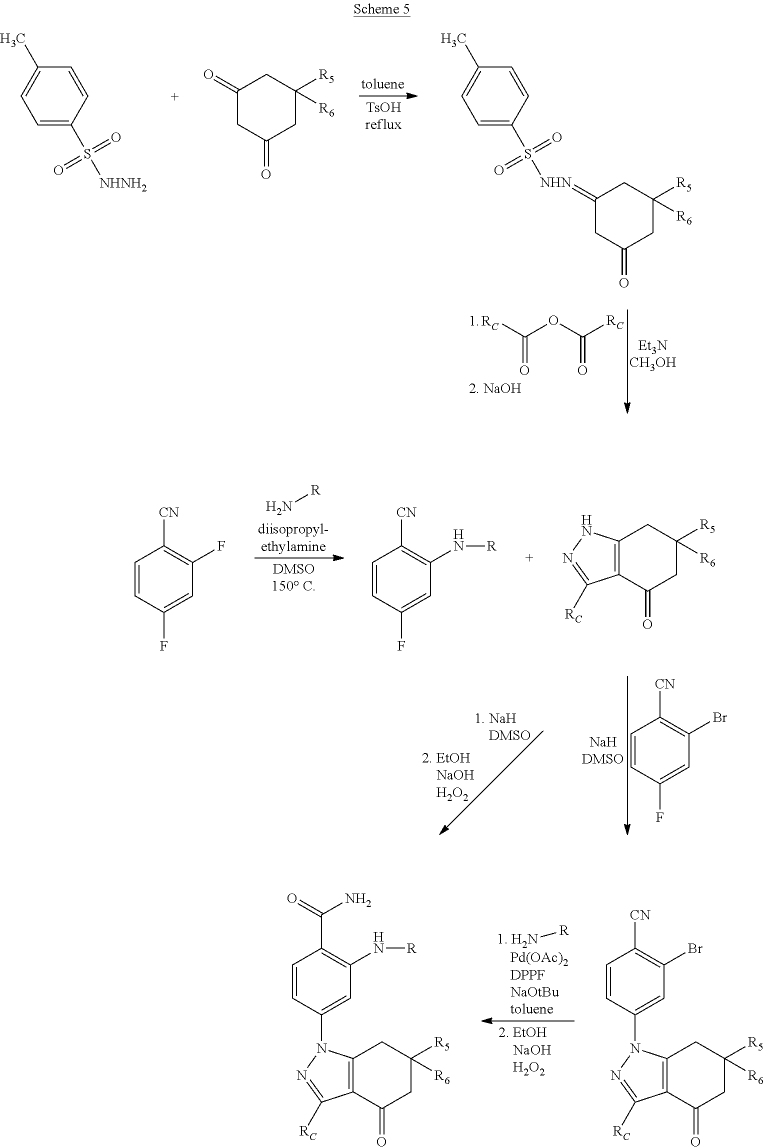

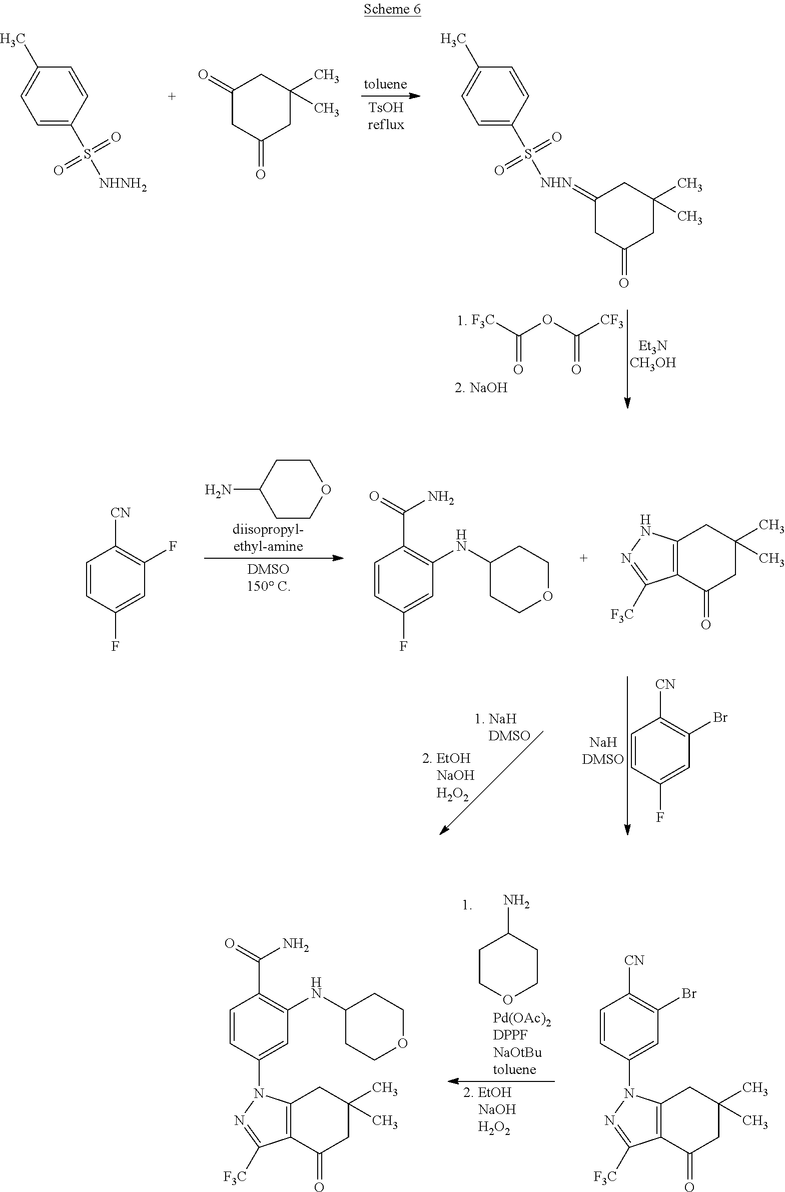

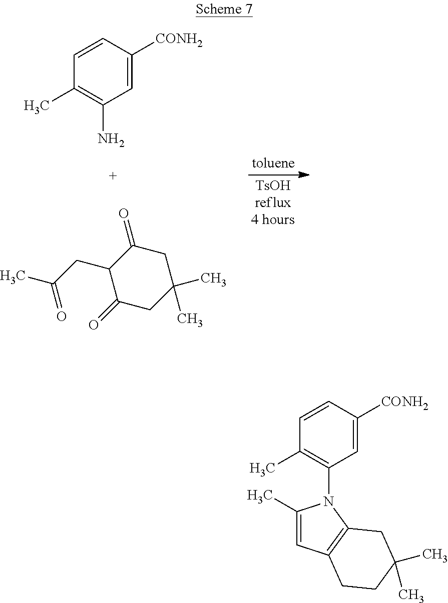

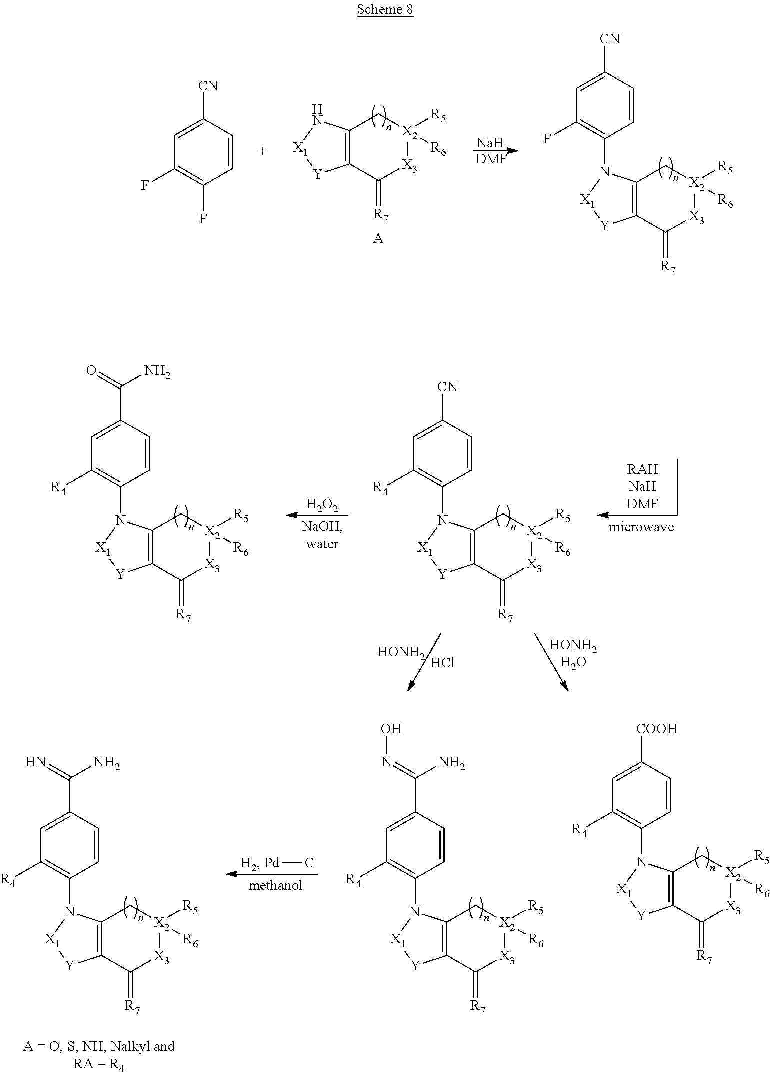

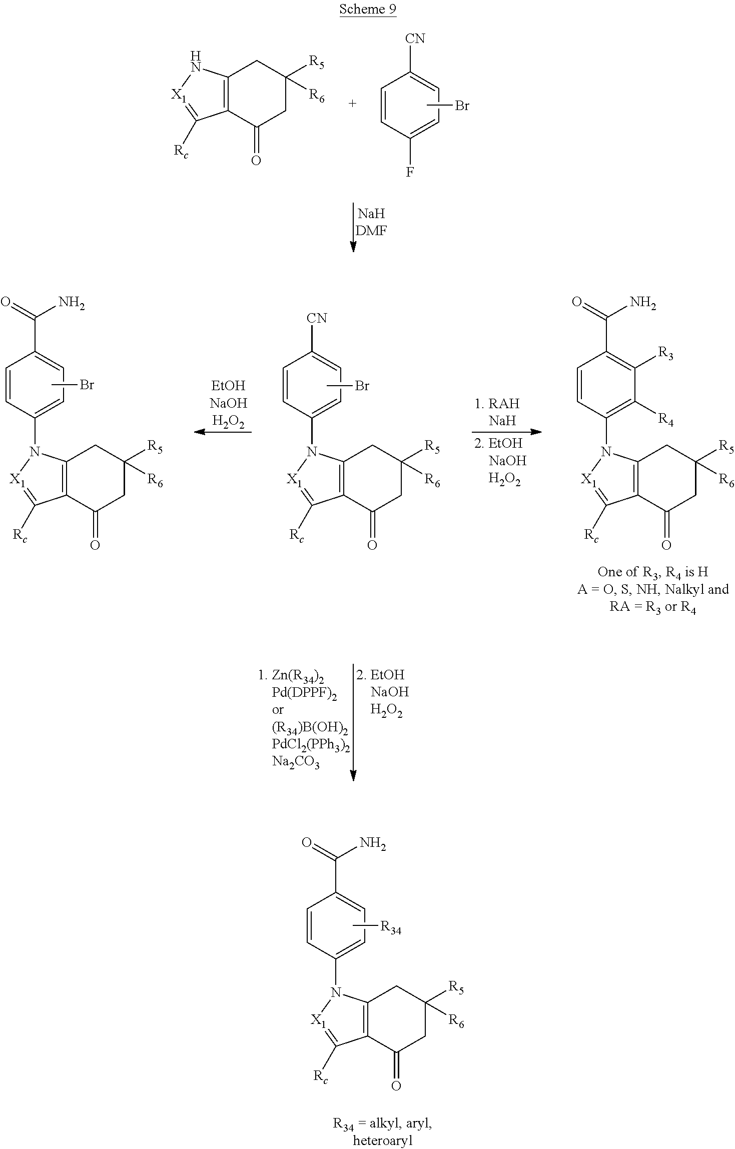

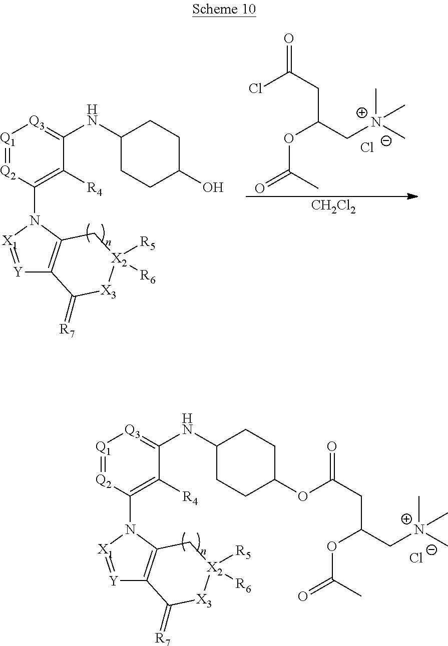

Those having skill in the art will recognize that the starting materials and reaction conditions may be varied, the sequence of the reactions altered, and additional steps employed to produce compounds encompassed by the present invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

### EXAMPLES

The preparation of the compounds of the invention is illustrated further by the following examples, which are not to be construed as limiting the invention in scope or spirit to the specific procedures and compounds described in them. In all cases, unless otherwise specified, the column chromatography is performed using a silica gel solid phase.

### Example 1

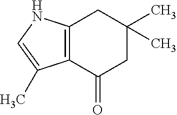

### 3,6,6-Trimethyl-1,5,6,7-tetrahydro-indo1-4-one (Intermediate 1)

To a solution of anti-pyruvic aldehyde-1-oxime (10 g, 1 eq) and 5,5-dimethyl-1,3-cyclohexanedione (16.1 g, 1 eq) in HOAc-H₂O (7:3, 200 mL) is added zinc powder (14.95 g, 2 eq) slowly with cooling by a water bath at room temperature. The mixture is then refluxed overnight, concentrated to dryness, partitioned between brine (300 mL) and dichloromethane (300 mL). The pH is adjusted to ca. 6 with saturated aqueous NaHCO₃, then the mixture is extracted with dichloromethane (3×200 mL). The organic layers are combined, dried over Na₂SO₄, filtered, concentrated. The crude product is purified by flash chromatography eluting with 5% ethyl acetate in dichloromethane. The combined organic fractions are concentrated, triturated in ether-hexane (2:1) for 1 hour, then filtered, washed with hexane to give the pure title compound (9 g, 45% yield) as a solid. LCMS m/z: (M+H)=178.1.

### Example 2

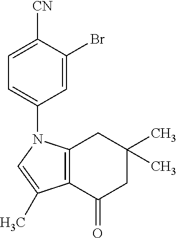 [SMILES: Cc1cn(-c2ccc(C#N)c(Br)c2)c2c1C(=O)CC(C)(C)C2]

2-Bromo-4-(3,6,6-trimethyl-4-oxo-4,5,6,7-tetrahydro-indol-1-yl)-benzonitrile (Intermediate 2)

The title compound of Example 1 (9.8 g, 55.3 mmol) and 2-bromo-4-fluorobenzonitrile (13.27 g, 66.4 mmol) are dissolved in anhydrous dimethylformamide (DMF, 300 mL). To this is added sodium hydride (95%, 2.79 g, 111 mmol) and the reaction is stirred at 55° C. for 1 hour. The reaction mixture is cooled to room temperature and water is added. A tan solid precipitated which is filtered, washed with water and ether and then dried in vacuo.

### Example 3

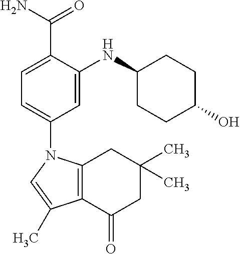 [SMILES: Cc1cn(-c2ccc(C(N)=O)c(N[C@H]3CC[C@H](O)CC3)c2)c2c1C(=O)CC(C)(C)C2]

2-(trans-4-Hydroxy-cyclohexylamino)-4-(3,6,6-trimethyl-4-oxo-4,5,6,7-tetrahydro-indol-1-yl)-benzamide (Intermediate 3)

A “Personal Chemistry” microwave vial is charged with the title compound of Example 2 [2-Bromo-4-(3,6,6-trimethyl-4-oxo-4,5,6,7-tetrahydro-indol-1-yl)-benzonitrile (1.072 g, 3.0 mmol)], trans-4-aminocyclohexanol (1.382 g, 12.0 mmol), palladium (II) acetate (33.7 mg, 5 mol %), 1,1′-bis(diphenylphosphino)ferrocene (DPPF) (166.3 mg, 10 mol %), and sodium tert-butoxide (576.7 mg, 6.0 mmol). To this is added toluene (20 mL) and the reaction is heated with microwave irradiation to 115° C. for 15 min. After allowing the reaction vessel to cool, a suspension formed and is filtered and the filtrate evaporated. The residue was purified by flash chromatography. The intermediate product is hydrolyzed by dissolution in 25% dimethylsulfoxide/ethanol, adding 0.5 mL of 1 N sodium hydroxide and 0.5 mL of 30% aqueous hydrogen peroxide, followed by stirring at room temperature for 4 hours. After judging the reaction to be complete by TLC, the DMSO/ethanol mixture is diluted with water and extracted with ethyl acetate (3×). The combined organics are washed with brine (2×), dried over Na₂SO₄, and evaporated. The compound is purified by column chromatography eluting with EtOAc-MeOH to yield 575 mg (47% yield) of the title compound as a white powder.

### Example 4

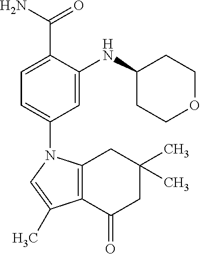

2-(Tetrahydro-pyran-4-ylamino)-4-(3,6,6-trimethyl-4-oxo-4,5,6,7-tetrahydro-indol-1-yl)-benzamide (Intermediate 4)

A “Personal Chemistry” microwave vial is charged with the title compound of Example 2 (2.858 g, 8.0 mmol), 4-aminotetrahydropyran (3.236 g, 32.0 mmol), palladium (II) acetate (89.8 mg, 5 mol %), 1,1′-bis(diphenylphosphino)ferrocene (443.6 mg, 10 mol %), and sodium tert-butoxide (1.538 g, 16.0 mmol). The reagents are suspended in toluene (40 mL) and are heated with microwave radiation to a temperature of 115° C. for fifteen minutes. After allowing the reaction vessel to cool, the suspension is filtered and the filtrate concentrated. After purifying the crude intermediate nitrile by flash chromatography, the nitrile is dissolved in 25% dimethylsulfoxide/ethanol, and 2 mL of 1 N sodium hydroxide and 2 mL of 30% aqueous hydrogen peroxide are added, followed by stirring at room temperature for 16 hours. The DMSO/ethanol mixture is then diluted with water and extracted with ethyl acetate (3×). The combined organics are washed with brine (2×), dried over Na₂SO₄, and evaporated. The residue is purified by column chromatography (EtOAc/MeOH) to yield 1.132 g (36%) of the title compound as an off-white powder.

### Example 5

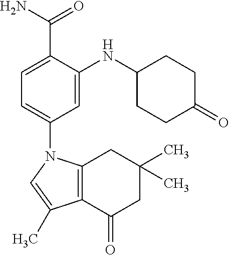 [SMILES: Cc1cn(-c2ccc(C(N)=O)c(NC3CCC(=O)CC3)c2)c2c1C(=O)CC(C)(C)C2]

### 2-(4-oxo-cyclohexylamino)-4-(3,6,6-trimethyl-4-oxo-4,5,6,7-tetrahydro-indo1-1-yl)-benzamide (Intermediate 5)

The title compound of Example 3 [2-(4-Hydroxy-cyclohexylamino)-4-(3,6,6-trimethyl-4-oxo-4,5,6,7-tetrahydro-indol-1-yl)-benzamide] (150 mg, 0.366 mmol) and Dess-Martin periodinane (0.366 mmol) are dissolved in anhydrous CH₂Cl₂ and stirred at room temperature for one hour. The reaction mixture is concentrated and the title compound is isolated as a white solid (36.2 mg, 24% yield) after purification by column chromatography eluting with EtOAc-MeOH.

### Example 6

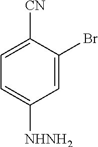 [SMILES: N#Cc1ccc(NN)cc1Br]

### 3-bromo-4-cyanophenylhydrazine (Intermediate 6)

In a clean, dry 250-mL round-bottom flask, 2-bromo-4-fluorobenzonitrile (25.34 g) is dissolved in tetrahydrofuran (50 mL) under N₂. To this is slowly added anhydrous hydrazine (50 mL). The solution color changed from yellow to red-orange. The reaction is allowed to stir at room temperature for 16 hours. A yellow-white crystalline solid precipitated from the solution. The mixture is then diluted with THF (50 mL) to dissolve the solids. The organic layer is then washed with saturated sodium bicarbonate solution until the pH of the organic layer is approximately 8.5. The organic layer is isolated and the solvent is removed under reduced pressure to give a white solid. This is placed in a fritted glass funnel and washed with 1.5 L of water, followed by of diethyl ether (ca. 200 mL). The ether wash is then combined with the white solid and dried under reduced pressure. The title compound is isolated as a fluffy, white or off-white solid (23.43 g, 87.2% yield).

### Example 6

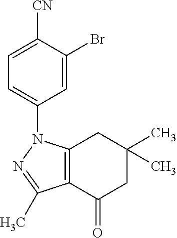 [SMILES: Cc1nn(-c2ccc(C#N)c(Br)c2)c2c1C(=O)CC(C)(C)C2]

### 2-Bromo-4-(3,6,6-trimethyl-4-oxo-4,5,6,7-tetrahydro-indazol-1-yl)-benzonitrile (Intermediate 7)

In a clean, dry 20 mL microwave reaction vial, the title compound of Example 10 (2.49 g) is combined with 2-acetyl-5,5-dimethyl-1,3-cyclohexanedione (2.14 g). The contents of the vial are dissolved in ethanol-acetic acid (12 mL, 3:1). The vial is sealed and agitated on a vortex. The vial is then placed in the microwave reactor and heated to 150° C. for 15 min. The vial is then cooled then placed in the refrigerator for 1 hour. The cooled solution is then diluted with water (8 mL) and poured onto a fritted glass funnel. The orange solid is washed with H₂O (100 mL) followed by ethanol (25 mL). The solid is then dried under reduced pressure. The title compound is obtained as of a light-orange crystalline solid (3.7463 g, 88.85% yield).

### Example 7

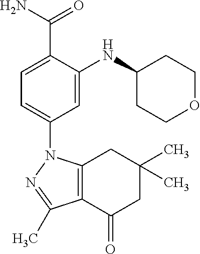

### 2-(Tetrahydro-pyran-4-ylamino)-4-(3,6,6-trimethyl-4-oxo-4,5,6,7-tetrahydro-indazol-1-yl)-benzamide (Intermediate 7A)

The title compound of Example 11 (100 mg, 0.28 mmol), Pd(OAc)₂ (3.2 mg, 5 mol %), DPPF (15.5 mg, 10 mol %) and NaO^{t}Bu (54 mg, 0.56 mmol) are added to a 2 mL microwave vial. Toluene (0.5 mL) and 4-aminotetrahydropyran (56 μL, 0.56 mmol) are added and the vial is evacuated and back-filled with N₂. The reaction mixture is heated at 120° C. for 15 min (microwave). The reaction mixture is filtered and the solids washed with methylene chloride. The product is purified using flash chromatography eluting with hexanes and ethyl acetate. Product is recovered as an off-white solid (88 mg, 83%), LCMS m/z: (M+H)=379.3. Ethanol (0.8 mL), DMSO (0.2 mL), NaOH (5 N, 93 μL, 2 mol eq) and H₂O₂ (0.1 mL, 30% solution in H₂O) are added to the pyrazole (88 mg, 0.23 mmol) in a 2 mL microwave vial. The reaction mixture is heated at 100° C. for 10 min. Product is recovered by washing with H₂O and ethyl acetate. Solvent is removed in vacuo to yield the title compound as a yellow solid (88 mg, 100%).

### Example 8

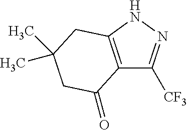 [SMILES: CC1(C)CC(=O)c2c(C(F)(F)F)n[nH]c2C1]

### 6,6-Dimethyl-3-trifluoromethyl-1,5,6,7-tetrahydro-indazol-4-one (Intermediate 8)

A mixture of 5,5-dimethyl-1,3-cyclohexandione (7.0 g, 49.9 mmol, 1.0 equiv), p-toluenesulfonylhydrazide (9.3 g, 49.9 mmol, 1.0 eq.), and p-toluenesulfonic acid (100 mg, 0.53 mmol, 0.01 eq.) in 300 mL of toluene is heated at reflux. After 30 minutes the reaction mixture is cooled, and 50 mL of toluene is added to the reaction mixture. The reaction is returned to heating at reflux. After 1 hour the reaction mixture is cooled to ambient temperature. The solids are collected by filtration, washed three times with ether, and dried under vacuum to afford 3,3-dimethyl-5-(p-tolylsulfonylhydrazono)-cyclohexanone (14.26 g, 93%) as a light yellow solid.

To a solution/suspension of 3,3-dimethyl-5-(p-tolylsulfonylhydrazono)-cyclohexanone (4.0g, 12.97 mmol, 1.0 eq.) in 72 ml of tetrahydrofuran and 24 mL of triethylamine is added trifluoroacetic anhydride (1.8 mL, 12.97 mmol, 1.0 eq.). The dark red reaction mixture is heated at 55° C. After 15 min the reaction mixture is homogeneous. After 2 h the reaction mixture is cooled to ambient temperature. Methanol (16 mL) and a 1:1 solution of water-1 M aqueous sodium hydroxide (16 mL) are added. After stirring for 3 h, the reaction mixture is diluted with 50 mL of saturated aqueous ammonium chloride and extracted with ethyl acetate (3×50 mL). The combined organic layers are washed with brine, dried over sodium sulfate, filtered, and concentrated in vacuo. The residue is filtered through a plug of silica gel, eluting with ethyl acetate. The filtrate is concentrated in vacuo, and the residue is treated with ether. The solids are collected by filtration and washed with ether. The filtrate is concentrated in vacuo, and the resulting residue is treated with ether. The solids are collected by filtration, washed with ether, and combined with the initial solids to provide 6,6-dimethyl-3-trifluoromethyl-1,5,6,7-tetrahydro-indazol-4-one (1.24 g, 41%) as a reddish orange solid.

### Example 8

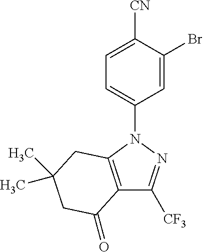 [SMILES: CC1(C)CC(=O)c2c(C(F)(F)F)nn(-c3ccc(C#N)c(Br)c3)c2C1]

### 2-bromo-4-(6,6-dimethyl-4-oxo-3-trifluoromethyl-4,5,6,7-tetrahydro-indazol-1-yl)-benzonitrile (Intermediate 9)

Sodium hydride (168 mg, 7.02 mmol, 1.0 eq.) is added to a solution of 6,6-dimethyl-3-trifluoromethyl-1,5,6,7-tetrahydro-indazol-4-one (1.63 g, 7.02 mmol, 1.0 eq.) in 35 mL of anhydrous dimethyl sulfoxide. After 15 min 2-bromo-4-fluorobenzonitrile (2.25 g, 11.23 mmol, 1.6 eq.) is added as a solid. The reaction mixture is heated at 45° C. After 23 h the reaction mixture is cooled to ambient temperature and quenched with 10 mL of saturated aqueous ammonium chloride. The mixture is diluted with water and extracted with ethyl acetate (4×). The combined organic layers are washed with brine, dried over sodium sulfate, filtered, and concentrated in vacuo. The residue is purified on a Biotage (SiO₂, hexanes-ethyl acetate) to afford 2-bromo-4-(6,6-dimethyl-4-oxo-3-trifluoromethyl -4,5,6,7-tetrahydro-indazol-1-yl)-benzonitrile (1.83 g, 63%) as an off-white powder.

### Example 9

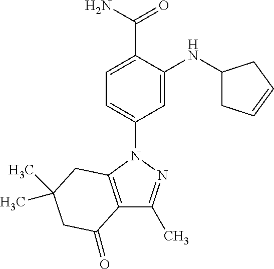 [SMILES: Cc1nn(-c2ccc(C(N)=O)c(NC3CC=CC3)c2)c2c1C(=O)CC(C)(C)C2]

### 2-(Cyclopent-3-enylamino)-4-(3,6,6-trimethyl-4-oxo-4,5,6,7-tetrahydro-indazol-1-yl)benzamide (Intermediate 9A)

1-(2-Bromo-4-cyanophen-4-yl)-3,6,6-trimethyltetrahydroindazal-4-one (2.0 g, 5.6 mmol), Pd(OAc)₂ (64 mg, 5 mol %), DPPF (328 mg, 10 mol %) and NaO^{t}Bu (1.13 mg, 11.2 mmol) are added to a 20 mL microwave vial. Toluene (15 mL) and 1-amino-3-cyclopentene (11.2 mmol) are added and the vial is evacuated and back-filled with N₂. The reaction mixture is heated at 120° C. for 15 minutes. The reaction mixture is filtered and the solids washed with CH₂Cl₂. The product is purified using flash chromatography eluting with hexanes and ethyl acetate. Product is obtained as an off-white solid (1.35 g).

The preceeding product (2.71g, 7.5 mmol) is dissolved in ethanol (20 mL) and DMSO (5 mL), and NaOH (5 N, 2.51 mL, 2 mol eq) and H₂O₂ (3.0 mL, 30% solution in H₂O) are added. The reaction mixture is stirred at room temperature for 2 hours. The reaction mixture is washed with H₂O and extracted with EtOAc. The product is purified using flash chromatography eluting with hexanes and ethyl acetate. The title compound is obtained as an off-white solid (490 mg).

### Example 10

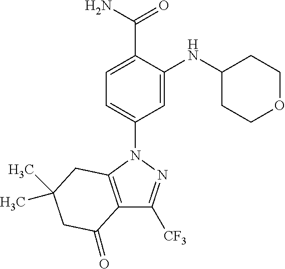 [SMILES: CC1(C)CC(=O)c2c(C(F)(F)F)nn(-c3ccc(C(N)=O)c(NC4CCOCC4)c3)c2C1]

### 4-(6,6-Dimethyl-4-oxo-3-trifluoromethyl-4,5,6,7-tetrahydro-indazol-1-yl) -2-(tetrahydro-pyran-4-ylamino)-benzamide (Intermediate 10)

4-Aminotetrahydropyran (245 mg, 2.42 mmol, 2.0 eq) and sodium tert-butoxide (233 mg, 2.42 mmol, 2.0 eq) are added to a stirred solution/suspension of 2-bromo-4-(6,6-dimethyl -4-oxo-3-trifluoromethyl-4,5,6,7-tetrahydro-indazol-1-yl)-benzonitrile (500 mg, 1.21 mmol, 1.0 eq), palladium (II) acetate (14 mg, 0.06 mmol, 0.05 eq), and DPPF (67 mg, 0.12 mmol, 0.10 eq) in 3.75 mL of toluene. The reaction vial is capped, and the reaction mixture is heated 20 minutes at 120° C. under microwave irradiation. The reaction mixture is diluted with water and ethyl acetate and combined. The layers are separated, and the aqueous layer is extracted with ethyl acetate (3×). The combined organic layers are washed with brine, dried over sodium sulfate, filtered, and concentrated in vacuo. The residue is purified on a Biotage system (SiO₂, hexanes-ethyl acetate) to afford 4-(6,6-dimethyl-4-oxo-3-trifluoromethyl -4,5,6,7-tetrahydro-indazol-1-yl)-2-(tetrahydro-pyran-4-ylamino)-benzonitrile (772 mg, 49%) as a tan solid: LC/MS (m/z): [M+H]⁺433.7. Further elution with ethyl acetate afforded 4-(6,6-dimethyl-4-oxo-3-trifluoromethyl-4,5,6,7-tetrahydro-indazol-1-yl)-2-(tetrahydro-pyran-4-ylamino)-benzamide (713 mg, 44%) as a yellow solid.

To a solution/suspension of 4-(6,6-dimethyl-4-oxo-3-trifluoromethyl-4,5,6,7-tetrahydro-indazol-1-yl)-2-(tetrahydro-pyran-4-ylamino)-benzonitrile (772 mg, 1.79 mmol, 1.0 eq) in 10 mL of 4:1 ethanol-dimethyl sulfoxide is added 1 mL of 1 M aqueous sodium hydroxide and 1 mL of 30% hydrogen peroxide. After 30 minutes the reaction mixture is diluted with water and extracted with ethyl acetate (4×). The combined organic layers are washed with brine, dried over sodium sulfate, filtered, and concentrated in vacuo to afford the title compound, 4-(6,6-dimethyl-4-oxo-3-trifluoromethyl-4,5,6,7-tetrahydro-indazol-1-yl) -2-(tetrahydro-pyran-4-ylamino)-benzamide (788 mg, 98%) as a tan solid.

### Example 11

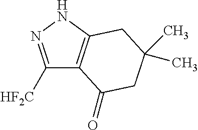

### 3-Difluoromethyl-6,6-dimethyl-1,5,6,7-tetrahydro-indazol-4-one (Intermediate 11)

A mixture of 5,5-Dimethyl-cyclohexane-1,3-dione (10 g), p-toluenesulfonylhydrazide (13.3 g) p-toluesulfonic acid (140 mg) in toluene (450 mL) is refluxed for 0.5 h. Then 100 mL of toluene is added and the mixture is refluxed for another 1 h. The mixture is cooled to room temperature, filtered, the solids are washed by ether (3×200 mL), and dried completely to give the hydrazone as a solid (20.4 g, 92.7%). It is used in the next step without further purification.

The preceeding product (8.86 g) is dissolved in a mixture of tetrahydrofuran (100 mL) and triethylamine (30 mL), placed under N₂, and difluoroacetic anhydride (5 g) is added slowly while swirling, then the mixture is heated to 55° C. overnight. The mixture is cooled to room temperature, and MeOH (35.5 mL) is added, followed by 35.5 mL of a 1:1 mixture of H₂O and 1 N NaOH. This mixture is stirred at room temperature for 3 h. The reaction mixture is diluted with saturated aqueous NH₄Cl (120 mL), extracted with ethyl aetate (4×150 mL), dried over Na₂SO₄, filtered, concentrated, purified by column chromatography eluting with a 1:1 mixture of ethyl acetate and hexanes to give 3-difluoromethyl-6,6-dimethyl-1,5,6,7-tetrahydro-indazol-4-one.

### Example 12

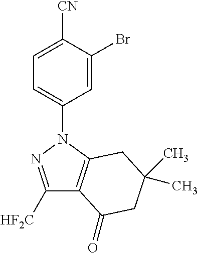

### 2-Bromo-4-(3-difluoromethyl-6,6-dimethyl-4-oxo-4,5,6,7-tetrahydro-indazol-1-yl)-benzonitrile (Intermediate 12)

NaH (350 mg) is added to a solution of 3-difluoromethyl-6,6-dimethyl-1,5,6,7-tetrahydro-indazol-4-one (3.12 g) in DMSO (75 mL) at room temperature. After 20 minutes of stirring, 2-bromo-4-fluorobenzonitrile (4.67 g) is added and stirred at 45° C. overnight. The reaction is diluted with saturated aqueous NH₄CI (100 mL), H₂O (100 mL). The mixture is extracted with ethyl acetate (4×150 mL), dried over Na₂SO₄, filtered, concentrated, purified by column chromatography eluting with a 1:2 mixture of ethyl acetate/hexanes. The concentrate of desired fractions is made into a slurry in ether, stirred for 2 h, filtered, washed by hexane to give pure solid 2-bromo-4-(6,6-dimethyl-4-oxo-3-difluoromethyl-4,5,6,7-tetrahydro-indazol-1-yl)-benzonitrile (2.82 g, 49.2%).

### Example 13

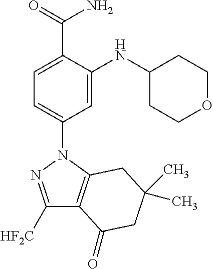

### 4-(6,6-Dimethyl-4-oxo-3-difluoromethyl-4,5,6,7-tetrahydro-indazol-1-yl)-2-(tetrahydro-pyran-4-ylamino)-benzamide (Intermediate 13)

The following reaction is conducted at the 300 mg scale and in duplicate at the 500 mg scale: 4-Aminotetrahydropyran (154 mg, 1.52 mmol, 2.0 eq.) and sodium tert-butoxide (146 mg, 1.52 mmol, 2.0 eq.) are added to a stirred solution/suspension of 2-bromo-4-(6,6-dimethyl-4-oxo-3-difluoromethyl-4,5,6,7-tetrahydro-indazol-1-yl)-benzonitrile (300 mg, 0.76 mmol, 1.0 eq.), palladium (II) acetate (8.5 mg, 0.04 mmol, 0.05 eq.), and 1,1′-bis(diphenylphosphino)ferrocene (42 mg, 0.08 mmol, 0.1 eq.) in toluene (2.25 mL). The reaction vial is capped, and the reaction mixture is heated for 20 min at 120° C. under microwave irradiation. The reaction mixture is diluted with water and ethyl acetate and combined. The layers are separated, and the aqueous layer is extracted with ethyl acetate (3×). The combined organic layers are washed with brine, dried over sodium sulfate, filtered, and concentrated in vacuo. The residue is purified on a Biotage system (SiO₂, hexanes-ethyl acetate) to afford 4-(6,6-dimethyl-4-oxo-3-difluoromethyl-4,5,6,7-tetrahydro-indazol-1-yl) -2-(tetrahydro-pyran-4-ylamino)-benzonitrile (897 mg, 65% yield) as a tan solid: LC/MS (m/z): [M+H]⁺415.2. Further elution with ethyl acetate afforded the title compound, 4-(6,6-dimethyl-4-oxo-3-difluoromethyl-4,5,6,7-tetrahydro-indazol-1-yl)-2-(tetrahydro-pyran-4-ylamino)-benzamide, (131 mg, 9%).

To a solution/suspension of 4-(6,6-dimethyl-4-oxo-3-difluoromethyl-4,5,6,7-tetrahydro-indazol-1-yl)-2-(tetrahydro-pyran-4-ylamino)-benzonitrile (897 mg, 2.16 mmol, 1.0 eq) in 10.8 mL of 4:1 mixture of ethanol-dimethyl sulfoxide is added 1 mL of 1 M aqueous sodium hydroxide and 1 mL of 30% hydrogen peroxide. After 30 minues the reaction mixture is diluted with water and extracted with ethyl acetate. The combined organic layers are washed with brine, dried over sodium sulfate, filtered, and concentrated in vacuo to afford the title compound, 4-(6,6-dimethyl-4-oxo-3-difluoromethyl-4,5,6,7-tetrahydro-indazol-1-yl) -2-(tetrahydro-pyran-4-ylamino)-benzamide, (869 mg, 93%) as a tan solid.

### Example 14

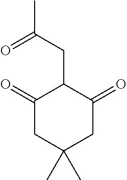

### 5,5-dimethyl-2-(2-oxopropyl)cyclohexane-1,3-dione (Intermediate 14)

An oven dried flask is charged with sodium hydride (3.61 g, 142.7 mmol) to which 200 mL of anhydrous DMF is added. The flask is cooled in an ice bath before adding 5,5-dimethyl-1,3-cyclohexanedione (20.0 g, 142.7 mmol) and chloroacetone (11.36 mL, 142.7 mmol) in 100 mL DMF in a controlled manner. The reaction is allowed to warm to RT and stirred for 3 h. Saturated NH₄Cl is added and the mixture is washed several times with EtOAc. The combined organic layer is dried over MgSO₄, filtered, the solvent removed in vacuo (below 40° C.).

### Example 15

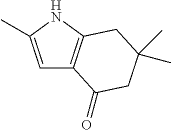

### 2,6,6-trimethyl-6,7-dihydro-1 H-indol-4(5H)-one (Intermediate 15)

The crude tri-ketone from Example 37 is dissolved in 300 mL of acetic acid to which ammonium acetate (55 g, 0.714 mmol) is added. The reaction mixture is heated at 65° C. until all starting material had disappeared (2-3 h), cooled to RT, added to H₂O, and washed with EtOAc. The organic layer is washed with saturated NaHCO₃ (×3), brine (×1) and dried over MgSO₄. Solvent is removed in vacuo and the oily residue is passed through a plug of silica. The appropriate fractions are collected and solvent is removed. The resulting solid is washed with EtOAc and hexanes (if too much hexanes is added the material crashing back out of solution after the addition of EtOAc will be gummy. To remedy this simply add a little EtOAc). The solid is filtered and washed with hexanes. More solid can be recovered by removing the solvent and repeating this procedure. LCMS m/z M+H=178.1. Approximately 5 g of a tan solid is collected and identified as 2,6,6-trimethyltetrahydroindol-4-one.

### Example 16

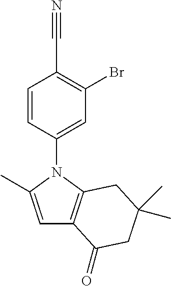

### 2-bromo-4-(2,6,6-trimethyl-4-oxo-4,5,6,7-tetrahydro-1H-indol-1-yl)benzonitrile (Intermediate 16)

Pyrrole (2,6,6-trimethyltetrahydroindol-4-one) (1.5 g, 8.5 mmol) and 2-bromo-4-fluorobenzonitrile (1.69 g, 8.5 mmol) are dissolved in anhydrous DMF (50 mL). To this NaH (95%, 408 mg, 17.0 mmol) is added and stirred at 50° C. for 1 h. The reaction mixture is cooled to RT and H₂O is added. Product crashed out of solution and is filtered and dried under vacuo (2.4 g, 79%).

### Example 17

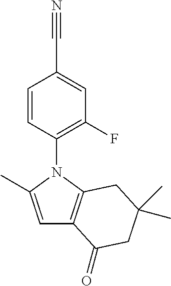

### 3-fluoro-4-(2,6,6-trimethyl-4-oxo-4,5,6,7-tetrahydro-1 H-indol-1-yl)benzonitrile (Intermediate 17)

Pyrrole (2,6,6-trimethyltetrahydroindol-4-one) (1.0 g, 5.6 mmol) and 3,4-difluorobenzonitrile (785 mg, 5.6 mmol) are dissolved in anhydrous DMF (20 mL). To this NaH (95%, 270 mg, 11.2 mmol) is added and stirred at 50° C. for 30 min. The reaction mixture is cooled to RT and washed with H₂O and EtOAc. The organic layer is dried over MgSO₄. Column chromatography on silica eluting with EtOAc-hexanes (1:1) gave the product as a yellow solid.

### Example 18

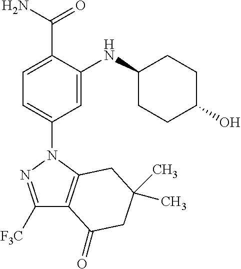 [SMILES: CC1(C)CC(=O)c2c(C(F)(F)F)nn(-c3ccc(C(N)=O)c(N[C@H]4CC[C@H](O)CC4)c3)c2C1]

### 4-(6,6-dimethyl-4-oxo-3-(trifluoromethyl)-4,5,6,7-tetrahydro-1H-indazol-1-yl)-2-((1r,4r)-4-hydroxycyclohexylamino)benzamide (Intermediate 19)

2-Bromo-4-(6, 6-dimethyl-4-oxo-3-trifl uoromethyl-4, 5, 6,7-tetrahyd ro-indazol-1-yl)-benzonitrile (60mg, 0.15mmol), Pd(OAc)₂ (1.7 mg, 5 mol %), DPPF (8.5 mg, 10 mol %) and NaO^{t}Bu (29.4 mg, 0.3 mmol) are added to a microwave vial. Toluene (0.5 mL) and trans-4-aminocyclohexanol (34 mg, 2 mol eq) are added and the vial is evacuated and back-filled with N₂. The reaction mixture is heated at 130° C. for 20 minutes (microwave). The reaction mixture is filtered and the solids washed with CH₂Cl₂. The product is purified using a Biotage SP1 Flash chromatography system (Biotage Si 12+M^{TM}, TLC Method, eluting with CH₂Cl₂ and CMA 80). The product pyrazole (10 mg, 2.2×10⁻⁵ mol) is dissolved in ethanol (0.8 mL) and DMSO (0.2mL) to which NaOH (5 N, 9 L, 2 mol eq) and H₂O₂ (excess, 30% solution in H₂O) is added. The reaction mixture is stirred at room temperature for 1 hour. The reaction mixture is washed with H₂O and extracted with EtOAc. The product is purified using a Biotage SP1 Flash chromatography system (Biotage Si 12+M^{TM}, TLC Method, eluting with CH₂Cl₂ and CMA 80). The title compound is obtained as an off-white solid.

### Example 19

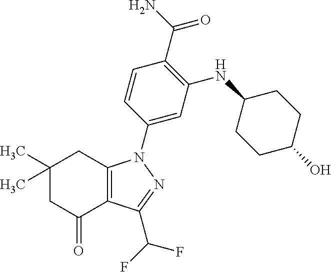 [SMILES: CC1(C)CC(=O)c2c(C(F)F)nn(-c3ccc(C(N)=O)c(N[C@H]4CC[C@H](O)CC4)c3)c2C1]

### 4-(3-(difluoromethyl)-6,6-dimethyl-4-oxo-4,5,6,7-tetrahydro-1H-indazol-1-yl)-2-(trans-4-hydroxycyclohexylamino)benzamide (Intermediate 20)

2-Bromo-4-(3-difluoromethyl-6,6-dimethyl-4-oxo-4,5,6,7-tetrahydro-indazol-1-yl)-benzonitrile (394.2 mg, 1.0 mmol), trans-4-aminocyclohexanol (288.0 mg mg, 2.5 mmol, 2.5 eq.), palladium (II) acetate (11.2 mg, 5 mol %), 1,1′-bis(diphenylphosphino)ferrocene (55.4 mg, 10 mol %), and sodium tert-butoxide (192.2 mg, 2.0 mmol, 2.0 eq.) are suspended in 4 mL of toluene. The reaction mixture is microwaved at 120° C. for 20 minutes. After cooling the reaction mixture, the solvent is removed in vacuo, and the residue diluted with water. The aqueous suspension is extracted with EtOAc (×3) and the combined organics washed with brine, dried over Na₂SO₄, and concentrated under reduced pressure. The intermediate nitrile and the desired amide (along with their O-acetyl adducts) are collected and combined after purifying by column chromatography (CH₂Cl₂/CMA 80), and are dissolved in 20 mL of 4:1 EtOH-DMSO. 1.2 mL of 1 N NaOH and 0.5 mL of 30% H₂O₂ are added, and the reaction mixture stirred at room temperature for 2 hours. The solution is diluted with water, extracted into EtOAc (×3), and the combined organics washed with brine (×2), dried over Na₂SO₄, and concentrated under reduced pressure. The residue is purified by column chromatography (EtOAc/MeOH), yielding 165.2 mg (37.0% yield) of the title compound as a pale yellow foam.

### Example 20

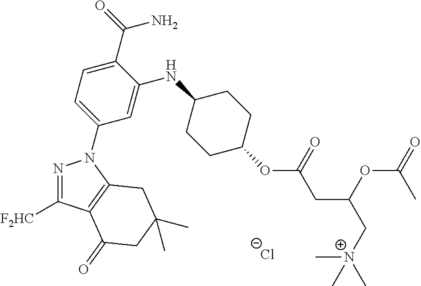 [SMILES: CC(=O)OC(CC(=O)O[C@H]1CC[C@H](Nc2cc(-n3nc(C(F)F)c4c3CC(C)(C)CC4=O)ccc2C(N)=O)CC1)C[N+](C)(C)C ; [Cl-]]

### 2-acetoxy-4-(((1r,4r)-4-((2-carbamoyl-5-(3-(difluoromethyl)-6,6-dimethyl-4-oxo-4,5,6,7-tetrahydro-1H-indazol-1-yl)phenyl)amino)cyclohexyl)oxy)-N,N,N-trimethyl-4-oxobutan-1-aminium chloride (Compound 1)

1. 2-acetoxy-4-chloro-N,N,N-trimethyl-4-oxobutan-1-aminium chloride may be prepared according to a procedure set forth in “Organic Chemistry”, 2006, vol. 26, pp. 946-949

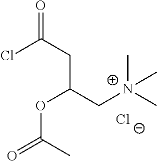

### (2-acetoxy-4-chloro-N,N,N-trimethyl-4-oxobutan-1-aminium chloride (Intermediate 21))

2. To 0.5 mmol of Intermediate 20 in 5 mL of CH₂Cl₂ is added 0.25 mmol of Intermediate 19. Stirring is continued overnight at room temperature. The reaction is quenched with aqueous sodium bicarbonate, and the product is then extracted with dichloromethane. The combined organic layers are washed with brine, and dried with anhydrous MgSO₄, and the solvent removed under reduced pressure to afford the title compound, 2-acetoxy-4-(((1r,4r)-4-((2-carbamoyl-5-(3-(difluoromethyl)-6,6-dimethyl-4-oxo -4,5,6,7-tetrahydro-1H-indazol-1-yl)phenyl)amino)cyclohexyl)oxy)-N, N, N-trimethyl-4-oxobutan-1-aminium chloride (Compound 1).

### Example 21

The following compounds are prepared essentially according to the procedures set forth in the above schemes and described in the above examples.

| Compound | | |
|---|---|---|
| No. | Structure | Name |
| 2 | 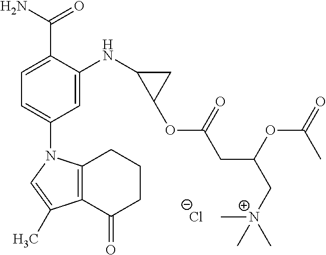 [SMILES: CC(=O)OC(CC(=O)OC1CC1Nc1cc(-n2cc(C)c3c2CCCC3=O)ccc1C(N)=O)C[N+](C)(C)C ; [Cl-]] | 2-acetoxy-4-(2-((2-carbamoyl-5-(3- methyl-4-oxo-4,5,6,7-tetrahydro-1H- indol-1-yl)phenyl)amino)cyclopropoxy)- N,N,N-trimethyl-4-oxobutan-1-aminium chloride |
| 3 | 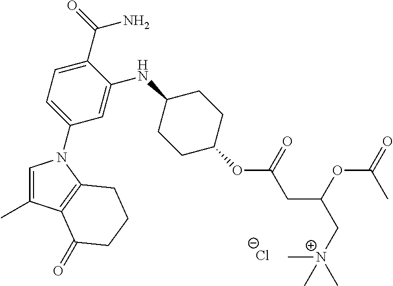 [SMILES: CC(=O)OC(CC(=O)O[C@H]1CC[C@H](Nc2cc(-n3cc(C)c4c3CCCC4=O)ccc2C(N)=O)CC1)C[N+](C)(C)C ; [Cl-]] | 2-acetoxy-4-(((1r,4r)-4-((2-carbamoyl- 5-(3-methyl-4-oxo-4,5,6,7-tetrahydro- 1H-indol-1- yl)phenyl)amino)cyclohexyl)oxy)- N,N,N-trimethyl-4-oxobutan-1-aminium chloride |
| 4 | 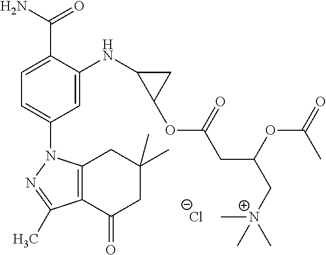 [SMILES: CC(=O)OC(CC(=O)OC1CC1Nc1cc(-n2nc(C)c3c2CC(C)(C)CC3=O)ccc1C(N)=O)C[N+](C)(C)C ; [Cl-]] | 2-acetoxy-4-(2-((2-carbamoyl-5-(3,6,6- trimethyl-4-oxo-4,5,6,7-tetrahydro-1H- indazol-1- yl)phenyl)amino)cyclopropoxy)-N,N,N- trimethyl-4-oxobutan-1-aminium chloride |
| 5 | 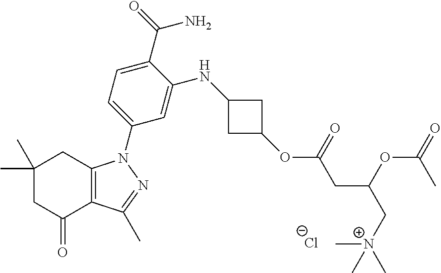 [SMILES: CC(=O)OC(CC(=O)OC1CC(Nc2cc(-n3nc(C)c4c3CC(C)(C)CC4=O)ccc2C(N)=O)C1)C[N+](C)(C)C ; [Cl-]] | 2-acetoxy-4-(3-((2-carbamoyl-5-(3,6,6- trimethyl-4-oxo-4,5,6,7-tetrahydro-1H- indazol-1- yl)phenyl)amino)cyclobutoxy)-N,N,N- trimethyl-4-oxobutan-1-aminium chloride |
| 6 | 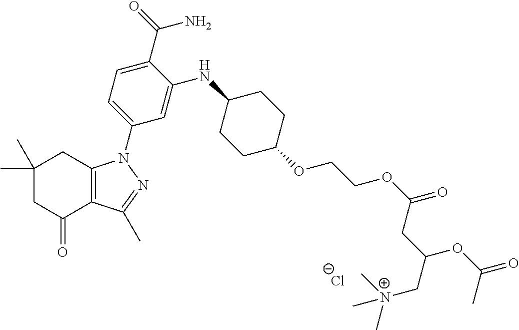 [SMILES: CC(=O)OC(CC(=O)OCCO[C@H]1CC[C@H](Nc2cc(-n3nc(C)c4c3CC(C)(C)CC4=O)ccc2C(N)=O)CC1)C[N+](C)(C)C ; [Cl-]] | 2-acetoxy-4-(2-(((1r,4r)-4-((2- carbamoyl-5-(3,6,6-trimethyl-4-oxo- 4,5,6,7-tetrahydro-1H-indazol-1- yl)phenyl)amino)cyclohexyl)oxy)ethoxy)- N,N,N-trimethyl-4-oxobutan-1- aminium chloride |
| 7 | 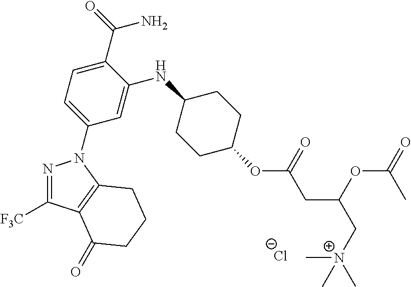 [SMILES: CC(=O)OC(CC(=O)O[C@H]1CC[C@H](Nc2cc(-n3nc(C(F)(F)F)c4c3CCCC4=O)ccc2C(N)=O)CC1)C[N+](C)(C)C ; [Cl-]] | 2-acetoxy-4-(((1r,4r)-4-((2-carbamoyl- 5-(4-oxo-3-(trifluoromethyl)-4,5,6,7- tetrahydro-1H-indazol-1- yl)phenyl)amino)cyclohexyl)oxy)- N,N,N-trimethyl-4-oxobutan-1-aminium chloride |
| 8 | 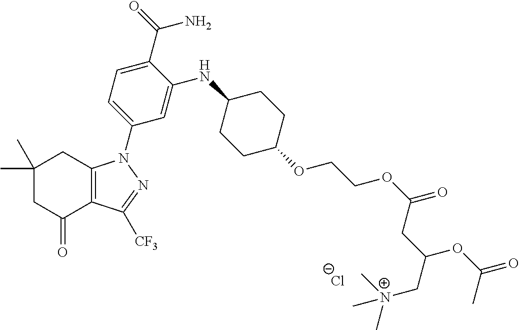 [SMILES: CC(=O)OC(CC(=O)OCCO[C@H]1CC[C@H](Nc2cc(-n3nc(C(F)(F)F)c4c3CC(C)(C)CC4=O)ccc2C(N)=O)CC1)C[N+](C)(C)C ; [Cl-]] | 2-acetoxy-4-(2-(((1r,4r)-4-((2- carbamoyl-5-(6,6-dimethyl-4-oxo-3- (trifluoromethyl)-4,5,6,7-tetrahydro-1H- indazol-1- yl)phenyl)amino)cyclohexyl)oxy)ethoxy)- N,N,N-trimethyl-4-oxobutan-1- aminium chloride |
| 9 | 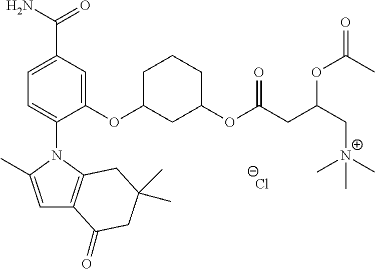 [SMILES: CC(=O)OC(CC(=O)OC1CCCC(Oc2cc(C(N)=O)ccc2-n2c(C)cc3c2CC(C)(C)CC3=O)C1)C[N+](C)(C)C ; [Cl-]] | 2-acetoxy-4-((3-(5-carbamoyl-2-(2,6,6- trimethyl-4-oxo-4,5,6,7-tetrahydro-1H- indol-1-yl)phenoxy)cyclohexyl)oxy)- N,N,N-trimethyl-4-oxobutan-1-aminium chloride; |
| 10 | 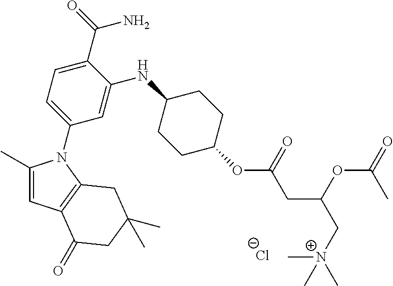 [SMILES: CC(=O)OC(CC(=O)O[C@H]1CC[C@H](Nc2cc(-n3c(C)cc4c3CC(C)(C)CC4=O)ccc2C(N)=O)CC1)C[N+](C)(C)C ; [Cl-]] | 2-acetoxy-4-(((1r,4r)-4-((2-carbamoyl- 5-(2,6,6-trimethyl-4-oxo-4,5,6,7- tetrahydro-1H-indol-1- yl)phenyl)amino)cyclohexyl)oxy)- N,N,N-trimethyl-4-oxobutan-1-aminium chloride; |
| 11 | 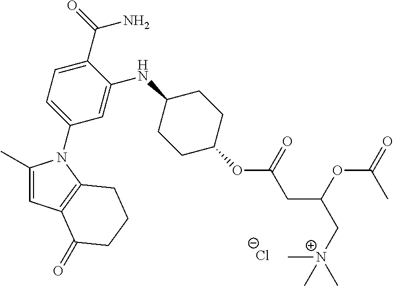 [SMILES: CC(=O)OC(CC(=O)O[C@H]1CC[C@H](Nc2cc(-n3c(C)cc4c3CCCC4=O)ccc2C(N)=O)CC1)C[N+](C)(C)C ; [Cl-]] | 2-acetoxy-4-(((1r,4r)-4-((2-carbamoyl- 5-(2-methyl-4-oxo-4,5,6,7-tetrahydro- 1H-indol-1- yl)phenyl)amino)cyclohexyl)oxy)- N,N,N-trimethyl-4-oxobutan-1-aminium chloride; |
| 12 | 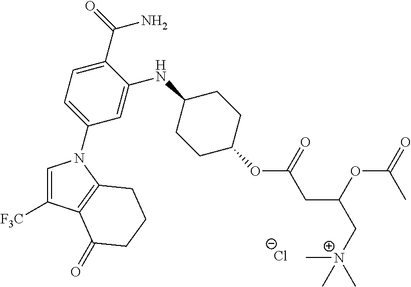 [SMILES: CC(=O)OC(CC(=O)O[C@H]1CC[C@H](Nc2cc(-n3cc(C(F)(F)F)c4c3CCCC4=O)ccc2C(N)=O)CC1)C[N+](C)(C)C ; [Cl-]] | 2-acetoxy-4-((1r,4r)-4-(2-carbamoyl-5- (4-oxo-3-(trifluoromethyl)-4,5,6,7- tetrahydro-1H-indol-1- yl)phenylamino)cyclohexyloxy)-N,N,N- trimethyl-4-oxobutan-1-aminium chloride |
| 13 | 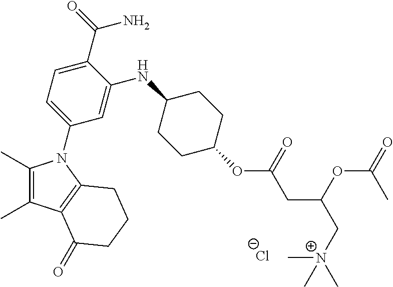 [SMILES: CC(=O)OC(CC(=O)O[C@H]1CC[C@H](Nc2cc(-n3c(C)c(C)c4c3CCCC4=O)ccc2C(N)=O)CC1)C[N+](C)(C)C ; [Cl-]] | 2-acetoxy-4-(((1r,4r)-4-((2-carbamoyl- 5-(2,3-dimethyl-4-oxo-4,5,6,7- tetrahydro-1H-indol-1- yl)phenyl)amino)cyclohexyl)oxy)- N,N,N-trimethyl-4-oxobutan-1-aminium chloride; |
| 14 | 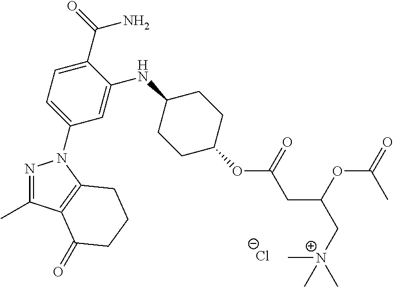 [SMILES: CC(=O)OC(CC(=O)O[C@H]1CC[C@H](Nc2cc(-n3nc(C)c4c3CCCC4=O)ccc2C(N)=O)CC1)C[N+](C)(C)C ; [Cl-]] | 2-acetoxy-4-(((1r,4r)-4-((2-carbamoyl- 5-(3-methyl-4-oxo-4,5,6,7-tetrahydro- 1H-indazol-1- yl)phenyl)amino)cyclohexyl)oxy)- N,N,N-trimethyl-4-oxobutan-1-aminium chloride |
| 15 | 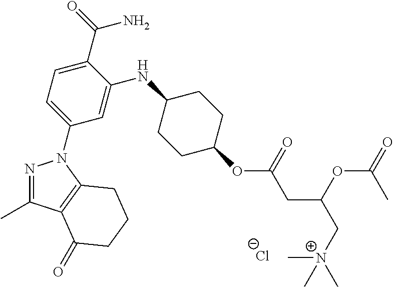 [SMILES: CC(=O)OC(CC(=O)O[C@H]1CC[C@@H](Nc2cc(-n3nc(C)c4c3CCCC4=O)ccc2C(N)=O)CC1)C[N+](C)(C)C ; [Cl-]] | 2-acetoxy-4-(((1s,4s)-4-((2-carbamoyl- 5-(3-methyl-4-oxo-4,5,6,7-tetrahydro- 1H-indazol-1- yl)phenyl)amino)cyclohexyl)oxy)- N,N,N-trimethyl-4-oxobutan-1-aminium chloride; |
| 16 | 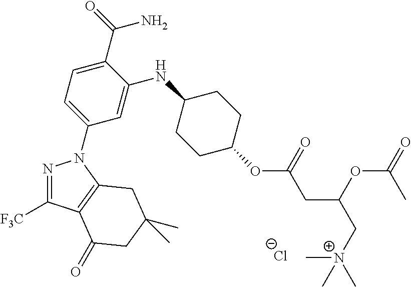 [SMILES: CC(=O)OC(CC(=O)O[C@H]1CC[C@H](Nc2cc(-n3nc(C(F)(F)F)c4c3CC(C)(C)CC4=O)ccc2C(N)=O)CC1)C[N+](C)(C)C ; [Cl-]] | 2-acetoxy-4-(((1r,4r)-4-((2-carbamoyl- 5-(6,6-dimethyl-4-oxo-3- (trifluoromethyl)-4,5,6,7-tetrahydro-1H- indazol-1- yl)phenyl)amino)cyclohexyl)oxy)- N,N,N-trimethyl-4-oxobutan-1-aminium chloride; |
| 17 | 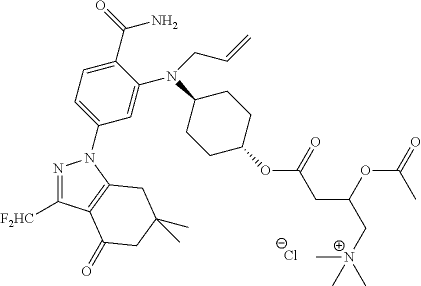 [SMILES: C=CCN(c1cc(-n2nc(C(F)F)c3c2CC(C)(C)CC3=O)ccc1C(N)=O)[C@H]1CC[C@H](OC(=O)CC(C[N+](C)(C)C)OC(C)=O)CC1 ; [Cl-]] | 2-acetoxy-4-((1r,4r)-4-(allyl(2- carbamoyl-5-(3-(difluoromethyl)-6,6- dimethyl-4-oxo-4,5,6,7-tetrahydro-1H- indazol-1- yl)phenyl)amino)cyclohexyloxy)-N,N,N- trimethyl-4-oxobutan-1-aminium chloride |
| 18 | 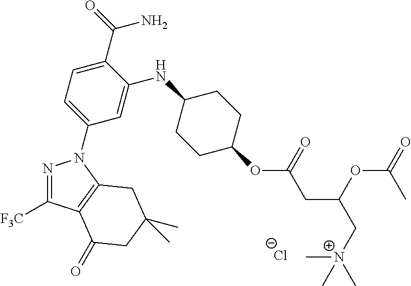 [SMILES: CC(=O)OC(CC(=O)O[C@H]1CC[C@@H](Nc2cc(-n3nc(C(F)(F)F)c4c3CC(C)(C)CC4=O)ccc2C(N)=O)CC1)C[N+](C)(C)C ; [Cl-]] | 2-acetoxy-4-(((1s,4s)-4-((2-carbamoyl- 5-(6,6-dimethyl-4-oxo-3- (trifluoromethyl)-4,5,6,7-tetrahydro-1H- indazol-1- yl)phenyl)amino)cyclohexyl)oxy)- N,N,N-trimethyl-4-oxobutan-1-aminium chloride; |
| 19 | 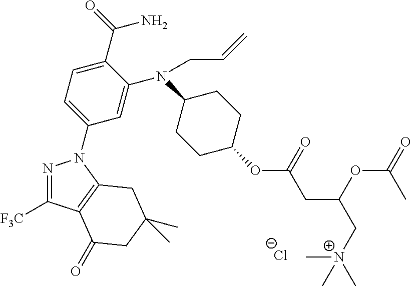 [SMILES: C=CCN(c1cc(-n2nc(C(F)(F)F)c3c2CC(C)(C)CC3=O)ccc1C(N)=O)[C@H]1CC[C@H](OC(=O)CC(C[N+](C)(C)C)OC(C)=O)CC1 ; [Cl-]] | 2-acetoxy-4-(((1r,4r)-4-(allyl(2- carbamoyl-5-(6,6-dimethyl-4-oxo-3- (trifluoromethyl)-4,5,6,7-tetrahydro-1H- indazol-1- yl)phenyl )amino)cyclohexyl)oxy)- N,N,N-trimethyl-4-oxobutan-1-aminium chloride; |
| 20 | 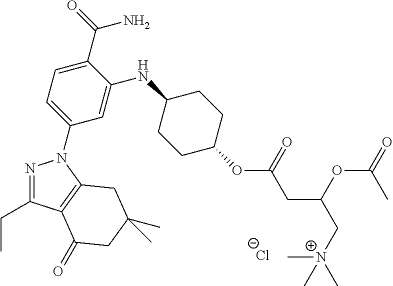 [SMILES: CCc1nn(-c2ccc(C(N)=O)c(N[C@H]3CC[C@H](OC(=O)CC(C[N+](C)(C)C)OC(C)=O)CC3)c2)c2c1C(=O)CC(C)(C)C2 ; [Cl-]] | 2-acetoxy-4-(((1r,4r)-4-((2-carbamoyl- 5-(3-ethyl-6,6-dimethyl-4-oxo-4,5,6,7- tetrahydro-1H-indazol-1- yl)phenyl)amino)cyclohexyl)oxy)- N,N,N-trimethyl-4-oxobutan-1-aminium chloride; |
| 21 | 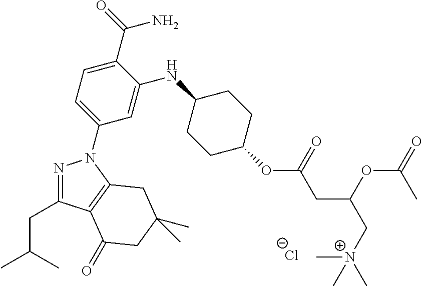 [SMILES: CC(=O)OC(CC(=O)O[C@H]1CC[C@H](Nc2cc(-n3nc(CC(C)C)c4c3CC(C)(C)CC4=O)ccc2C(N)=O)CC1)C[N+](C)(C)C ; [Cl-]] | 2-acetoxy-4-(((1r,4r)-4-((2-carbamoyl- 5-(3-isobutyl-6,6-dimethyl-4-oxo- 4,5,6,7-tetrahydro-1H-indazol-1- yl)phenyl)amino)cyclohexyl)oxy)- N,N,N-trimethyl-4-oxobutan-1-aminium chloride; |
| 22 | 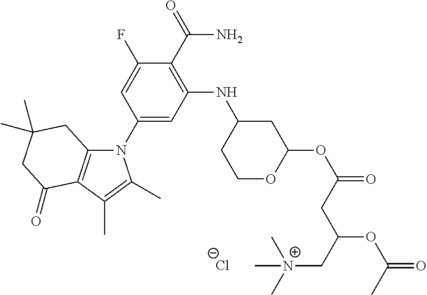 [SMILES: CC(=O)OC(CC(=O)OC1CC(Nc2cc(-n3c(C)c(C)c4c3CC(C)(C)CC4=O)cc(F)c2C(N)=O)CCO1)C[N+](C)(C)C ; [Cl-]] | 2-acetoxy-4-((4-((2-carbamoyl- 3-fluoro-5-(2,3,6,6-tetramethyl-4-oxo- 4,5,6,7-tetrahydro-1H-indol-1- yl)phenyl)amino)tetrahydro-2H-pyran- 2-yl)oxy)-N,N,N-trimethyl-4-oxobutan- 1-aminium chloride; |
| 23 | 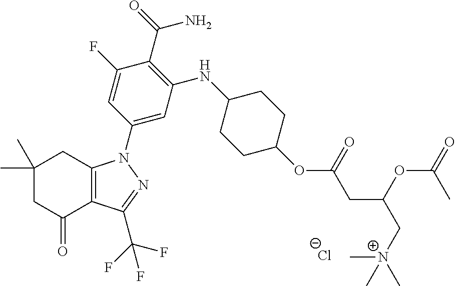 [SMILES: CC(=O)OC(CC(=O)OC1CCC(Nc2cc(-n3nc(C(F)(F)F)c4c3CC(C)(C)CC4=O)cc(F)c2C(N)=O)CC1)C[N+](C)(C)C ; [Cl-]] | 2-acetoxy-4-((4-((2-carbamoyl- 5-(6,6-dimethyl-4-oxo-3- (trifluoromethyl)-4,5,6,7-tetrahydro-1H- indazol-1-yl)-3- fluorophenyl)amino)cyclohexyl)oxy)- N,N,N-trimethyl-4-oxobutan-1-aminium chloride |
| 24 | 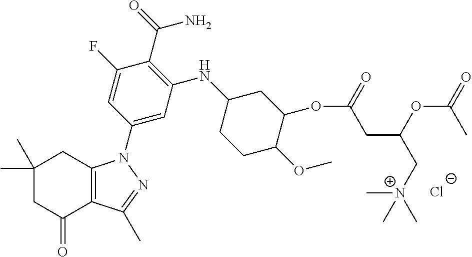 [SMILES: COC1CCC(Nc2cc(-n3nc(C)c4c3CC(C)(C)CC4=O)cc(F)c2C(N)=O)CC1OC(=O)CC(C[N+](C)(C)C)OC(C)=O ; [Cl-]] | 2-acetoxy-4-((5-((2-carbamoyl- 3-fluoro-5-(3,6,6-trimethyl-4-oxo- 4,5,6,7-tetrahydro-1H-indazol-1- yl)phenyl)amino)-2-methoxy- cyclohexyl)oxy)-N,N,N-trimethyl- 4-oxobutan-1-aminium chloride; |
| 25 | 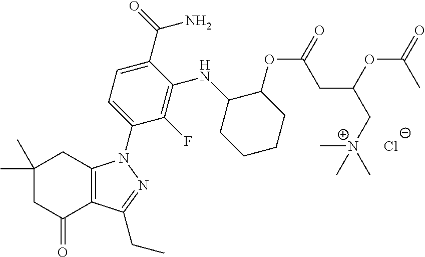 [SMILES: CCc1nn(-c2ccc(C(N)=O)c(NC3CCCCC3OC(=O)CC(C[N+](C)(C)C)OC(C)=O)c2F)c2c1C(=O)CC(C)(C)C2 ; [Cl-]] | 2-acetoxy-4-((2-((6-carbamoyl- 3-(3-ethyl-6,6-dimethyl-4-oxo-4,5,6,7- tetrahydro-1H-indazol-1-yl)-2- fluorophenyl)amino)cyclohexyl)oxy)- N,N,N-trimethyl-4-oxobutan-1-aminium chloride; |
| 26 | 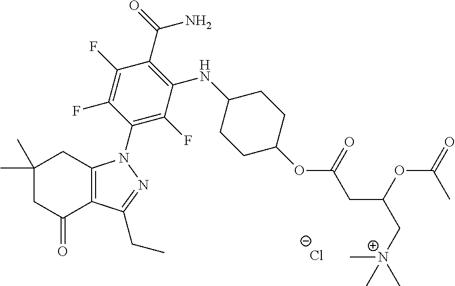 [SMILES: CCc1nn(-c2c(F)c(F)c(C(N)=O)c(NC3CCC(OC(=O)CC(C[N+](C)(C)C)OC(C)=O)CC3)c2F)c2c1C(=O)CC(C)(C)C2 ; [Cl-]] | 2-acetoxy-4-((4-((2-carbamoyl- 5-(3-ethyl-6,6-dimethyl-4-oxo-4,5,6,7- tetrahydro-1H-indazol-1-yl)-3,4,6- trifluorophenyl)amino)cyclohexyl)oxy)- N,N,N-trimethyl-4-oxobutan-1-aminium chloride; |
| 27 | 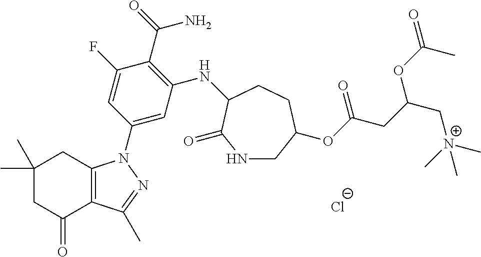 | 2-acetoxy-4-((6-((2-carbamoyl- 3-fluoro-5-(3,6,6-trimethyl-4-oxo- 4,5,6,7-tetrahydro-1H-indazol-1- yl)phenyl)amino)-7-oxoazepan-3- yl)oxy)-N,N,N-trimethyl-4-oxobutan- 1-aminium chloride; |
| 28 | 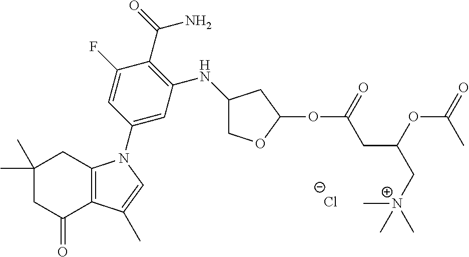 [SMILES: CC(=O)OC(CC(=O)OC1CC(Nc2cc(-n3cc(C)c4c3CC(C)(C)CC4=O)cc(F)c2C(N)=O)CO1)C[N+](C)(C)C ; [Cl-]] | 2-acetoxy-4-((4-((2-carbamoyl- 3-fluoro-5-(3,6,6-trimethyl-4-oxo- 4,5,6,7-tetrahydro-1H-indol-1- yl)phenyl)amino)tetrahydrofuran-2- yl)oxy)-N,N,N-trimethyl-4-oxobutan- 1-aminium chloride; |
| 29 | 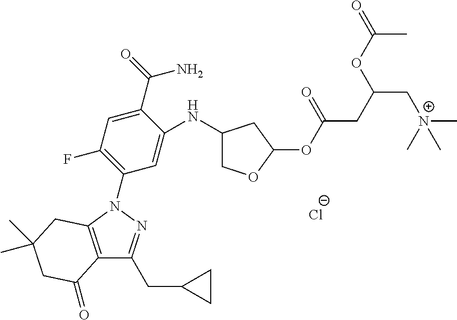 | 2-acetoxy-4-((4-((2-carbamoyl- 5-(3-(cyclopropylmethyl)-6,6-dimethyl- 4-oxo-4,5,6,7-tetrahydro-1H-indazol-1- yl)-4-fluorophenyl)amino)tetrahydro- furan-2-yl)oxy)-N,N,N-trimethyl-4- oxobutan-1-aminium chloride; |
| 30 | 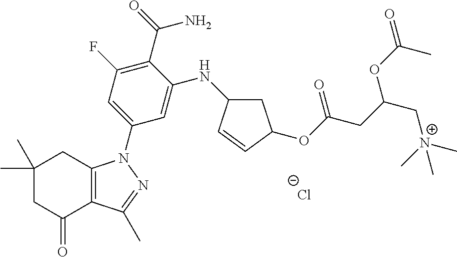 | 2-acetoxy-4-((4-((2-carbamoyl- 3-fluoro-5-(3,6,6-trimethyl-4-oxo- 4,5,6,7-tetrahydro-1H-indazol-1- yl)phenyl)amino)cyclopent-2-en-1- yl)oxy)-N,N,N-trimethyl-4-oxobutan-1- aminium chloride; |
| 31 | 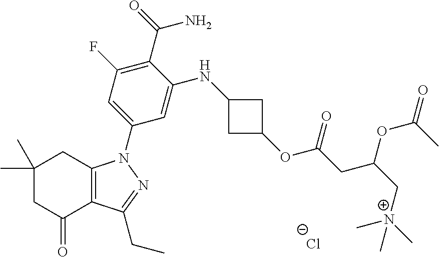 [SMILES: CCc1nn(-c2cc(F)c(C(N)=O)c(NC3CC(OC(=O)CC(C[N+](C)(C)C)OC(C)=O)C3)c2)c2c1C(=O)CC(C)(C)C2 ; [Cl-]] | 2-acetoxy-4-(3-((2-carbamoyl- 5-(3-ethyl-6,6-dimethyl-4-oxo-4,5,6,7- tetrahydro-1H-indazol-1-yl)-3- fluorophenyl)amino)cyclobutoxy)- N,N,N-trimethyl-4-oxobutan-1-aminium chloride; |
| 32 | 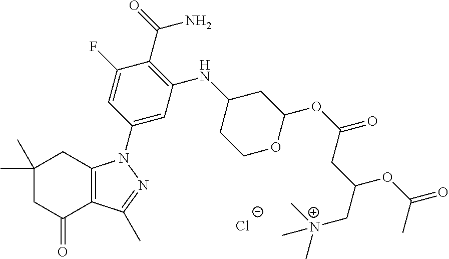 [SMILES: CC(=O)OC(CC(=O)OC1CC(Nc2cc(-n3nc(C)c4c3CC(C)(C)CC4=O)cc(F)c2C(N)=O)CCO1)C[N+](C)(C)C ; [Cl-]] | 2-acetoxy-4-((4-((2-carbamoyl- 3-fluoro-5-(3,6,6-trimethyl-4-oxo- 4,5,6,7-tetrahydro-1H-indazol-1- yl)phenyl)amino)tetrahydro-2H-pyran- 2-yl)oxy)-N,N,N-trimethyl-4-oxobutan- 1-aminium chloride; |
| 33 | 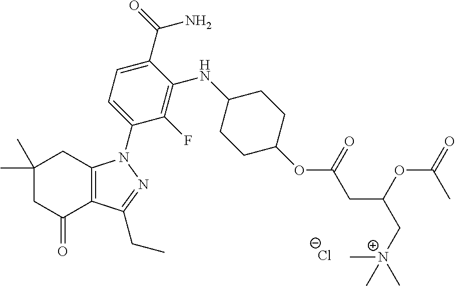 [SMILES: CCc1nn(-c2ccc(C(N)=O)c(NC3CCC(OC(=O)CC(C[N+](C)(C)C)OC(C)=O)CC3)c2F)c2c1C(=O)CC(C)(C)C2 ; [Cl-]] | 2-acetoxy-4-((4-((6-carbamoyl- 3-(3-ethyl-6,6-dimethyl-4-oxo-4,5,6,7- tetrahydro-1H-indazol-1-yl)-2- fluorophenyl)amino)cyclohexyl)oxy)- N,N,N-trimethyl-4-oxobutan-1-aminium chloride; |
| 34 | 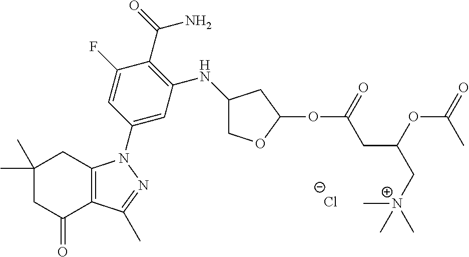 [SMILES: CC(=O)OC(CC(=O)OC1CC(Nc2cc(-n3nc(C)c4c3CC(C)(C)CC4=O)cc(F)c2C(N)=O)CO1)C[N+](C)(C)C ; [Cl-]] | 2-acetoxy-4-((4-((2-carbamoyl- 3-fluoro-5-(3,6,6-trimethyl-4-oxo- 4,5,6,7-tetrahydro-1H-indazol-1- yl)phenyl)amino)tetrahydrofuran-2- yl)oxy)-N,N,N-trimethyl-4-oxobutan-1- aminium chloride; |
| 35 | 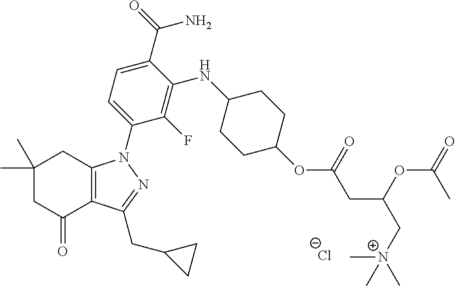 [SMILES: CC(=O)OC(CC(=O)OC1CCC(Nc2c(C(N)=O)ccc(-n3nc(CC4CC4)c4c3CC(C)(C)CC4=O)c2F)CC1)C[N+](C)(C)C ; [Cl-]] | 2-acetoxy-4-((4-((6-carbamoyl- 3-(3-(cyclopropylmethyl)-6,6-dimethyl- 4-oxo-4,5,6,7-tetrahydro-1H-indazol-1- yl)-2-fluorophenyl)amino)cyclo- hexyl)oxy)-N,N,N-trimethyl-4- oxobutan-1-aminium chloride; |
| 36 | 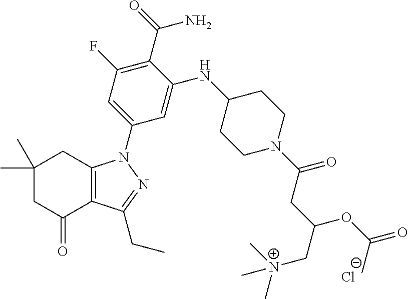 [SMILES: CCc1nn(-c2cc(F)c(C(N)=O)c(NC3CCN(C(=O)CC(C[N+](C)(C)C)OC(C)=O)CC3)c2)c2c1C(=O)CC(C)(C)C2 ; [Cl-]] | 2-acetoxy-4-(4-((2-carbamoyl- 5-(3-ethyl-6,6-dimethyl-4-oxo-4,5,6,7- tetrahydro-1H-indazol-1-yl)-3- fluorophenyl)amino)piperidin-1-yl)- N,N,N-trimethyl-4-oxobutan-1- aminium chloride |
| 37 | 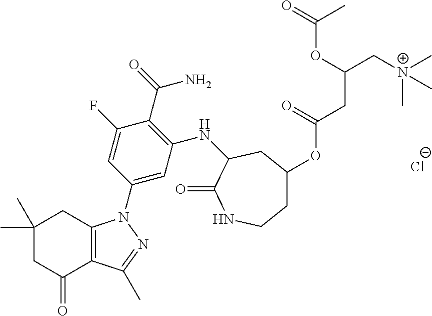 | 2-acetoxy-4-((6-((2-carbamoyl-3-fluoro- 5-(3,6,6-trimethyl-4-oxo-4,5,6,7- tetrahydro-1H-indazol-1- yl)phenyl)amino)-7-oxoazepan-4- yl)oxy)-N,N,N-trimethyl-4-oxobutan-1- aminium chloride |
| 38 | 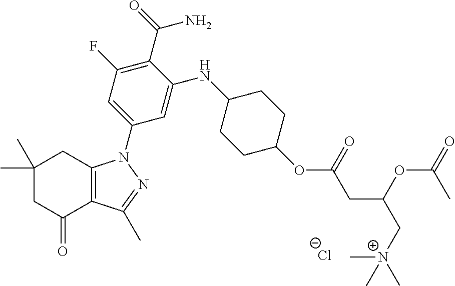 [SMILES: CC(=O)OC(CC(=O)OC1CCC(Nc2cc(-n3nc(C)c4c3CC(C)(C)CC4=O)cc(F)c2C(N)=O)CC1)C[N+](C)(C)C ; [Cl-]] | 2-acetoxy-4-((4-((2-carbamoyl- 3-fluoro-5-(3,6,6-trimethyl-4-oxo- 4,5,6,7-tetrahydro-1H-indazol-1- yl)phenyl)amino)cyclohexyl)oxy)- N,N,N-trimethyl-4-oxobutan-1-aminium chloride; |
| 39 | 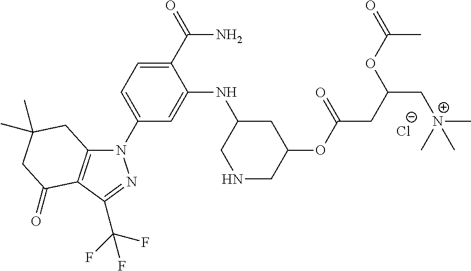 | 4-(6,6-dimethyl-4-oxo-3- (trifluoromethyl)-4,5,6,7-tetrahydro-1H- indazol-1-yl)-2-(piperidin-3- ylamino)benzamide; |
| 40 | 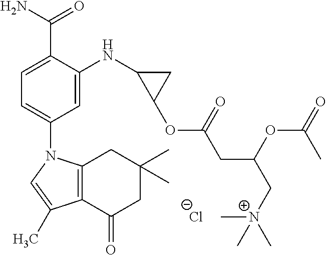 [SMILES: CC(=O)OC(CC(=O)OC1CC1Nc1cc(-n2cc(C)c3c2CC(C)(C)CC3=O)ccc1C(N)=O)C[N+](C)(C)C ; [Cl-]] | 2-acetoxy-4-(2-((2-carbamoyl-5-(3,6,6- trimethyl-4-oxo-4,5,6,7-tetrahydro-1H- indol-1-yl)phenyl)amino)cyclopropoxy)- N,N,N-trimethyl-4-oxobutan-1-aminium chloride |
| 41 | 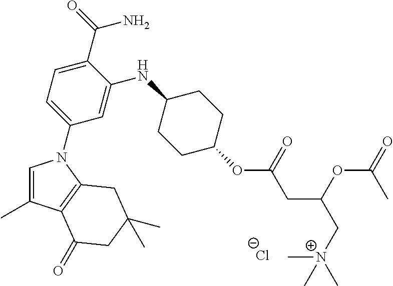 [SMILES: CC(=O)OC(CC(=O)O[C@H]1CC[C@H](Nc2cc(-n3cc(C)c4c3CC(C)(C)CC4=O)ccc2C(N)=O)CC1)C[N+](C)(C)C ; [Cl-]] | 2-acetoxy-4-(((1r,4r)-4-((2-carbamoyl- 5-(3,6,6-trimethyl-4-oxo-4,5,6,7- tetrahydro-1H-indol-1- yl)phenyl)amino)cyclohexyl)oxy)- N,N,N-trimethyl-4-oxobutan-1-aminium chloride |
| 42 | 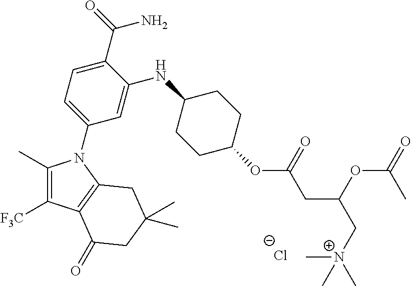 [SMILES: CC(=O)OC(CC(=O)O[C@H]1CC[C@H](Nc2cc(-n3c(C)c(C(F)(F)F)c4c3CC(C)(C)CC4=O)ccc2C(N)=O)CC1)C[N+](C)(C)C ; [Cl-]] | 2-acetoxy-4-((1r,4r)-4-(2-carbamoyl-5- (2,6,6-trimethyl-4-oxo-3- (trifluoromethyl)-4,5,6,7-tetrahydro-1H- indol-1-yl)phenylamino)cyclohexyloxy)- N,N,N-trimethyl-4-oxobutan-1-aminium chloride |
| 43 | 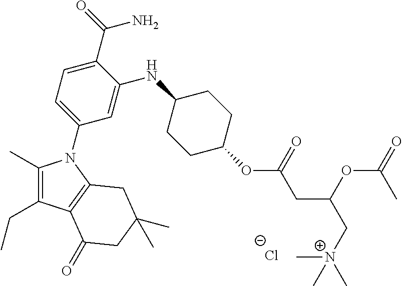 [SMILES: CCc1c2c(n(-c3ccc(C(N)=O)c(N[C@H]4CC[C@H](OC(=O)CC(C[N+](C)(C)C)OC(C)=O)CC4)c3)c1C)CC(C)(C)CC2=O ; [Cl-]] | 2-acetoxy-4-((1r,4r)-4-(2-carbamoyl-5- (3-ethyl-2,6,6-trimethyl-4-oxo-4,5,6,7- tetrahydro-1H-indol-1- yl)phenylamino)cyclohexyloxy)-N,N,N- trimethyl-4-oxobutan-1-aminium chloride |
| 44 | 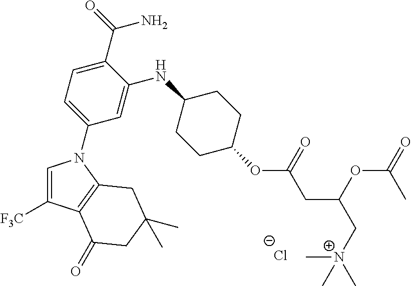 [SMILES: CC(=O)OC(CC(=O)O[C@H]1CC[C@H](Nc2cc(-n3cc(C(F)(F)F)c4c3CC(C)(C)CC4=O)ccc2C(N)=O)CC1)C[N+](C)(C)C ; [Cl-]] | 2-acetoxy-4-((1r,4r)-4-(2-carbamoyl-5- (6,6-dimethyl-4-oxo-3-(trifluoromethyl)- 4,5,6,7-tetrahydro-1H-indol-1- yl)phenylamino)cyclohexyloxy)-N,N,N- trimethyl-4-oxobutan-1-aminium chloride |
| 45 | 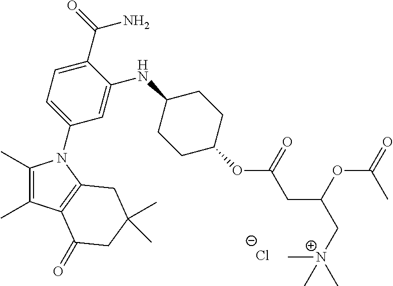 [SMILES: CC(=O)OC(CC(=O)O[C@H]1CC[C@H](Nc2cc(-n3c(C)c(C)c4c3CC(C)(C)CC4=O)ccc2C(N)=O)CC1)C[N+](C)(C)C ; [Cl-]] | 2-acetoxy-4-(((1r,4r)-4-((2-carbamoyl- 5-(2,3,6,6-tetramethyl-4-oxo-4,5,6,7- tetrahydro-1H-indol-1- yl)phenyl)amino)cyclohexyl)oxy)- N,N,N-trimethyl-4-oxobutan-1-aminium chloride |
| 46 | 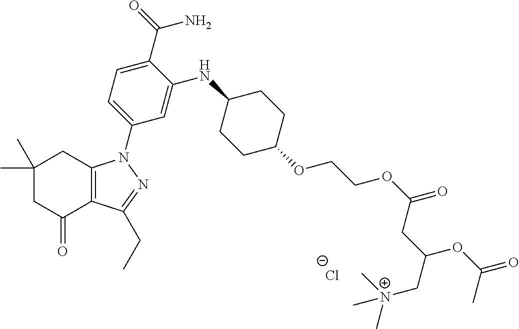 [SMILES: CCc1nn(-c2ccc(C(N)=O)c(N[C@H]3CC[C@H](OCCOC(=O)CC(C[N+](C)(C)C)OC(C)=O)CC3)c2)c2c1C(=O)CC(C)(C)C2 ; [Cl-]] | 2-acetoxy-4-(2-((1r,4r)-4-(2-carbamoyl- 5-(3-ethyl-6,6-dimethyl-4-oxo-4,5,6,7- tetrahydro-1H-indazol-1- yl)phenylamino)cyclohexyloxy)ethoxy)- N,N,N-trimethyl-4-oxobutan-1-aminium chloride |
| 47 | 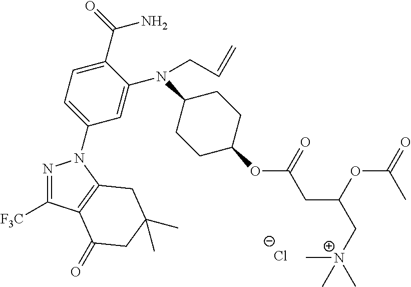 [SMILES: C=CCN(c1cc(-n2nc(C(F)(F)F)c3c2CC(C)(C)CC3=O)ccc1C(N)=O)[C@H]1CC[C@@H](OC(=O)CC(C[N+](C)(C)C)OC(C)=O)CC1 ; [Cl-]] | 2-acetoxy-4-((1s,4s)-4-(allyl(2- carbamoyl-5-(6,6-dimethyl-4-oxo-3- (trifluoromethyl)-4,5,6,7-tetrahydro-1H- indazol-1-yl)phenyl)amino)cyclo- hexyloxy)-N,N,N-trimethyl-4- oxobutan-1-aminium chloride |

### Biological Evaluation

### Example 22

### Cell Proliferation Assays

A panel of cancer cell lines may be obtained from the DCTP Tumor Repository, National Cancer Institute (Frederick, MD) or ATCC (Rockville, MD). Cell cultures are maintained in Hyclone RPMI 1640 medium (Logan, UT) supplemented with 10% fetal bovine serum and 20 mM HEPES buffer, final pH 7.2, at 37° C. with a 5% CO₂ atmosphere. Cultures are maintained at sub-confluent densities. Human umbilical vein endothelial cells (HUVEC) are purchased from Clonetics, a division of Cambrex (Walkersville, Md.). Cultures are established from cryopreserved stocks using Clonetics EGM-2 medium supplemented with 20 mM HEPES, final pH 7.2, at 37° C. with a 5% CO₂ atmosphere.

For proliferation assays, cells are seeded with the appropriate medium into 96 well plates at 1,000-2,500 cells per well, depending on the cell line, and are incubated overnight. The following day, test compound, DMSO solution (negative control), or Actinomycin D (positive control) is added to the appropriate wells as 10× concentrated stocks prepared in phosphate buffered saline. The cell plates are then incubated for an additional 2-5 days, depending on the cell line, to allow proliferation to occur. To measure cell density, 50 μL of WST-1 solution (Roche Applied Science, IN) diluted 1:5 in phosphate buffered saline is added to each well, and the cells incubated for an additional 1-5 hrs., again depending on the cell line. Optical density is determined for each well at 450 nM using a Tecan GeniosPro plate reader (RTP, NC). The percentage of cell growth is determined by comparing the cell growth in the presence of test compounds to the cells treated with DMSO vehicle (control, 100% growth) and cells treated with Actinomycin D (10 μM, 0% growth).

Immediately after the WST-1 determination, the medium is removed from the PC-3, NCl-H460 and HUVEC cell lines, and the plates stored at −80° C. Using these assay plates, relative amounts of DNA in each well are determined using the Cyquant DNA assay kit from R&D Systems (Eugene, Oreg.) following the manufacturer's directions. Results for each compound treatment are compared to DMSO vehicle control (100%) and 10 μM Actinomycin D treated cells (0%).

### Example 23

### Determination of Affinity for HSP-90

### Heat Shock Protein 90

Affinity of test compounds for HSP-90 is determined as follows: Protein mixtures obtained from a variety of organ tissues (for example: spleen, liver and lung) are reversibly bound to a purine affinity column to capture purine-binding proteins, especially HSP-90. The purine affinity column is washed several times, and then eluted with 20 μM, 100 μM, and 500 μM of test compound. Compounds of Formula I elute HP-90 in a dose-dependent manner vs. a control elution using dimethylsulfoxide. The elution profile of Formula I compounds is determined by 1-dimensional SDS polyacrylamide gel electrophoresis. Gels are stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels are imaged using a standard flat bed gel imager and the amount of protein estimated by densitometry. The percent of HSP-90 protein eluted from the column at each concentration is determined and IC₅₀ values are calculated from these estimates. The identity of a band containing HSP-90 is determined by protein sequencing using mass spectroscopy.

Compounds of the invention are inhibitors of HSP-90 (heat shock protein 90).

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

1. A compound of the formula, 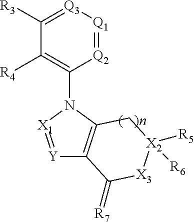
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S-(C₁-C₆)alkyl, —SO₂-(C₁-C₆)alkyl, —SO₂-aryl, —SO-(C₁-C₆)alkyl, —SO-aryl, —SO₂NH₂, —SO₂NH-(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, —SO₂N -(C₁-C₆)alkyl, —SO₂NH-aryl, —SO₂-aryl, -SO-(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₃, wherein
Z is OR₀ or —N(R₃₀)₂, wherein
each R₃₀ is independently —H or C₁-C₆ alkyl, or N(R₃ₒ)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
Rₒ is —H, -C₁-C₁₀ alkyl, -C₂-C₁₀ alkenyl, -C₂-C₁₀ alkynyl, aryl, heteroaryl, or -C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and the R₂₃ groups are optionally substituted with at least one group which independently is hydroxy, oxo, halo, amino, cyano, nitro, —SH, —S-(C₁-C₆)alkyl, —SO₂-(C₁-C₆)alkyl, —SO₂-aryl, —SO-(C₁-C₆)alkyl, —SO-aryl, —SO₂NH₂, —SO₂NH-(C₁-C₆)alkyl, —SO₂NH-aryl, (C₁-C₆)alkoxy, or mono- or di-(C₁-C₁₀)alkylamino; and
wherein at least one of R₃ and R₄ is substituted with a group R₅₀ where R₅₀ is: 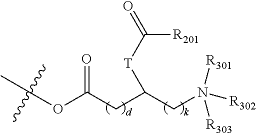
wherein
d and k are integers independently selected from 1 and 2;
R₂₀₁ is (C₁-C₆)alkyl where the alkyl is optionally substituted with (C₃-C₇)cycloalkyl, (C₂-C₆) alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH-(C₁-C₆ alkyl), N-O-(C₀-C₆)alkyl-R₂₂, N-(C₁-C₆ alkenoxy);or N-(0₁-C₆ alkoxy optionally substituted with carboxy);
Y is N or CR_{S}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-Cᵢₒ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, cyano, nitro, halo(C₁-C₆)alkyl, halo(C₁-C₆)alkoxy, carboxamide, heterocycloalkyl, aryl, or heteroaryl,
wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, or carboxamide;
R_{C′} is -C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, -C₁-C₁₀ alkyl, -C₁-C₁₀ -aloalkyl, -C₃-C₇ cycloalkyl, -heterocycloalkyl, -C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
X₁ is N or CR_{C};
Q1, Q2, and Q₃ are independently N or CR_{Q} , wherein one and only one of Q₁, Q₂, and Q₃ is C-R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 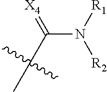
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN});
and
X₄ is O, S, NH, NOH, N—NH₂, N—NHaryl, N—NH-(C₁-C₆ alkyl), or N-(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide,
wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then
R₆ is absent and R₅ is H or C₁-C₆ alkyl;
(iii) X₃ is C, then
it is substituted with two groups that are independently H, C₁-C₆ alkyl, or mono- or di-(C₁-C₆)alkylamino(C₁-C₆)alkyl; and
(iv) X₂ is 0 or S(O)ₚ, then R₆ and R₅ are absent.

2. A compound or salt according to claim 1, wherein R₃ is substituted with R₅₀, and R₅₀ is

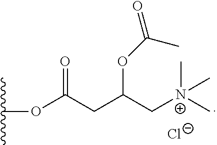 [SMILES: *OC(=O)CC(C[N+](C)(C)C)OC(C)=O ; [Cl-]]

3-5. (canceled)

6. A compound according to any of the preceding claims where X₁ is N.

7. A compound according to any of the preceding claims where X₁ is CR_{C}.

8-9. (canceled)

10. A compound or salt according to claim 1, wherein
Q3 is CR₂₁, wherein
R₂₁ is a group of the formula, 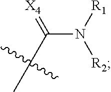
R₃ is —Z₁R_{Z1}, wherein
Z₁ is —O— or —NH—; and
R_{Z1} is a saturated or unsaturated C₃-C₁₀ cycloalkyl, each of which is
(a) substituted with R₅₀, where R₅₀ is 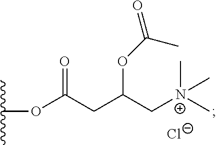 [SMILES: *OC(=O)CC(C[N+](C)(C)C)OC(C)=O ; [Cl-]]
and
(b) optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, —SO₂NH-(C₁-C₆)alkyl, —SO₂NH-aryl, —SO₂-aryl, —SO -(C₁-C₆)alkyl, —SO₂-aryl, C₁-C₆ alkoxy, C₂-C₁₀ alkenyloxy, C₂-C₁₀ alkynyloxy, mono- or di-(C₁-C₁ₒ)alkylamino, —OCᵢ-Cᵢₒ alkyl-Z, or R₂₃; and
R₄ is H or halogen.

11. A compound or salt according to claim 10, wherein R_{Z1} is a saturated C₅-C₇ cycloalkyl.

12. A compound or salt according to claim 10, wherein R_{Z1} is a unsaturated C₅-C₇ cycloalkyl.

13. A compound according to claim 10 where X₁ is N.

14. A compound according to claim 10 where X₁ is CR_{C}.

15. A compound or salt according to claim 14, wherein X₁ is CH.

16. A compound or salt according to claim 15, wherein
R₁ and R₂ are independently H or C₁-C₄ alkyl;
Q₁ and Q₂ are both CH;
X₂ is C substituted with two independently selected C₁-C₄ alkyl groups;
and
n is 1.

17. A compound or salt according to claim 1, of the formula, 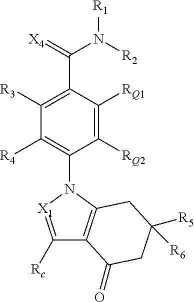
wherein
R_{Q1} is H or halogen; and
R_{Q2} is H or halogen.

18. A compound or salt according to claim 17, wherein
R₃ is cyclohexyl which is
(a) substituted with R₅₀, where R₅₀ is 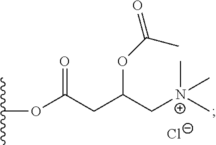 [SMILES: *OC(=O)CC(C[N+](C)(C)C)OC(C)=O ; [Cl-]]
and
(b) optionally substituted at any available position with 0₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S-(C₁-C₆)alkyl, —SO₂-(C₁-C₆)alkyl, —SO₂NH₂, -SO₂NH-(C₁-C₆)alkyl, —SO₂NH-aryl, —SO₂-aryl, —SO-(C₁-C₆)alkyl, —SO₂-aryl, C₁-C₆ alkoxy, C₂-C₁₀ alkenyloxy, C₂-C₁₀ alkynyloxy, mono- or di-(C₁-C₁ₒ)alkylamino, —OC₁-Cᵢo alkyl-Z, or R₂₃; and
R₄ is H or fluoro.

19. A compound according to claim 18 where X₁ is N.

20. A compound according to claim 18 where X₁ is CH.

21. A compound which is:
2-acetoxy-4-(((1r,4r)-4-(2-carbamoyl-5-(3-(difluoromethyl)-6,6-dimethyl-4-oxo-4,5,6,7-tetrahydro-1H-indazol-1-yl)phenyl)amino)cyclohexyl)oxy)-N,N,N-trimethyl-4-oxobutan-1-aminium chloride;
2-acetoxy-4-(2-((2-carbamoyl-5-(3-methyl-4-oxo-4,5,6,7-tetrahydro -1H-indol-1-yl)phenyl)amino)cyclopropoxy)-N,N,N-trimethyl-4-oxobutan-1-aminium chloride;
2-acetoxy-4-(((1r,4r)-4-((2-carbamoyl-5-(3-methyl-4-oxo-4,5,6,7-tetrahydro-1H-indol-1-yl)phenyl)amino)cyclohexyl)oxy)-N,N,N-trimethyl-4-oxobutan-1-aminium chloride;
2-acetoxy-4-(2-((2-carbamoyl-5-(3,6,6-trimethyl-4-oxo-4,5,6,7-tetrahydro-1H-indazol-1-yl)phenyl)amino)cyclopropoxy)-N,N,N-trimethyl-4-oxobutan-1-aminium chloride;
2-acetoxy-4-(3-((2-carbamoyl-5-(3,6,6-trimethyl-4-oxo-4,5,6,7-tetrahydro-1H-indazol-1-yl)phenyl)amino)cyclobutoxy)-N,N,N-trimethyl-4-oxobutan-1-aminium chloride;
2-acetoxy-4-(2-(((1r,4r)-4-((2-carbamoyl-5-(3,6,6-trimethyl-4-oxo -4,5,6,7-tetrahydro-1H-indazol-1-yl)phenyl)amino)cyclohexyl)oxy)ethoxy) -N,N,N-trimethyl-4-oxobutan-1-aminium chloride;
2-acetoxy-4-(((1r,4)-4-((2-carbamoyl-5-(4-oxo-3-(trifluoromethyl) -4,5,6,7-tetrahydro-1H-indazol-1-yl)phenyl)amino)cyclohexyl)oxy)-N,N,N -trimethyl-4-oxobutan-1-aminium chloride;
2-acetoxy-4-(2-(((1r,4) -4-((2-carbamoyl-5-(6,6-dimethyl-4-oxo-3-(trifluoromethyl)-4,5,6,7-tetrahydro-1H-indazol-1-yl)phenyl)amino)cyclohexyl)oxy)ethoxy)-N,N,N-trimethyl-4-oxobutan-1-aminium chloride;
2-acetoxy-4-((3-(5-carbamoyl-2-(2,6,6-trimethyl-4-oxo-4,5,6,7-tetrahydro-1H-indol-1-yl)phenoxy)cyclohexyl)oxy)-N,N,N-trimethyl-4-oxobutan-1-aminium chloride;
2-acetoxy-4-(((1r,4r)-4-((2-carbamoyl-5-(2,6,6-trimethyl-4-oxo -4,5,6,7-tetrahydro-1H-indol-1-yl)phenyl)amino)cyclohexyl)oxy)-N,N,N -trimethyl-4-oxobutan-1-aminium chloride;
2-acetoxy-4-(((1 r,4r)-4-((2-carbamoyl-5-(2-methyl-4-oxo-4,5,6,7-tetrahydro-1H-indol-1-yl)phenyl)amino)cyclohexyl)oxy)-N,N,N-trimethyl-4-oxobutan-1-aminium chloride;
2-acetoxy-4-((1r,4r)-4-(2-carbamoyl-5-(4-oxo-3-(trifluoromethyl) -4,5,6,7-tetrahydro-1H-indol-1-yl)phenylamino)cyclohexyloxy)-N,N,N -trimethyl-4-oxobutan-1-aminium chloride;
2-acetoxy-4-(((1 r,4r)-4-((2-carbamoyl-5-(2,3-dimethyl-4-oxo-4,5,6,7-tetrahydro-1H-indol-1-yl)phenyl)amino)cyclohexyl)oxy)-N,N,N-trimethyl-4-oxobutan-1-aminium chloride;
2-acetoxy-4-(((1 r,4r)-4-((2-carbamoyl-5-(3-methyl-4-oxo-4,5,6,7-tetrahydro-1H-indazol-1-yl)phenyl)amino)cyclohexyl)oxy)-N,N,N-trimethyl -4-oxobutan-1-aminium chloride;
2-acetoxy-4-(((1 s,4s)-4-((2-carbamoyl-5-(3-methyl-4-oxo-4,5,6,7-tetrahydro-1H-indazol-1-yl)phenyl)amino)cyclohexyl)oxy)-N,N,N-trimethyl -4-oxobutan-1-aminium chloride;
2-acetoxy-4-(((1r,4r)-4-((2-carbamoyl-5-(6,6-dimethyl-4-oxo-3-(trifluoromethyl)-4,5,6,7-tetrahydro-1H-indazol-1-yl)phenyl)amino)cyclohexyl)oxy)-N,N,N-trimethyl-4-oxobutan-1-aminium chloride;
2-acetoxy-4-((1r,4r)-4-(allyl(2-carbamoyl-5-(3-(difluoromethyl)-6,6-dimethyl-4-oxo-4,5,6,7-tetrahydro-1H-indazol-1-yl)phenyl)amino)cyclohexyloxy)-N,N,N-trimethyl-4-oxobutan-1-aminium chloride;
2-acetoxy-4-(((1s,4s)-4-((2-carbamoyl-5-(6,6-dimethyl-4-oxo-3-(trifluoromethyl)-4,5,6,7-tetrahydro-1 H-indazol-1 -yl)phenyl)amino)cyclohexyl)oxy)-N,N,N-trimethyl-4-oxobutan-1 -aminium chloride;
2-acetoxy-4-(((1r,4r)-4-(allyl(2-carba moyl-5-(6,6-d imethyl-4-oxo-3-(trifluoromethyl)-4,5,6,7-tetrahydro-1 H-indazol-1 -yl)phenyl)amino)cyclohexyl)oxy)-N,N,N-trimethyl-4-oxobutan-1 -aminium chloride;
2-acetoxy-4-(((1 r,4r)-4-((2-carbamoyl-5-(3-ethyl-6,6-d imethyl-4-oxo -4,5,6,7-tetrahydro-1 H-indazol-1 -yl)phenyl)amino)cyclohexyl)oxy)-N,N, N-trimethyl-4-oxobutan-1 -aminium chloride;
2-acetoxy-4-(((1r,4r)-4-((2-carbamoyl-5-(3-isobutyl-6,6-dimethyl-4-oxo-4,5,6,7-tetrahydro-1 H-indazol-1 -yl)phenyl)amino)cyclohexyl)oxy) -N,N,N-trimethyl-4-oxobutan-1 -aminium chloride;
2-acetoxy-4-((4-((2-carbamoyl-3-fluoro-5-(2,3,6,6-tetramethyl-4-oxo -4,5,6,7-tetrahydro-1 H-indol-1 -yl)phenyl)amino)tetrahydro-2H-pyran-2-yl)oxy)-N,N,N-trimethyl-4-oxobutan-1 -aminium chloride;
2-acetoxy-4-((4-((2-carbamoyl-5-(6,6-dimethyl-4-oxo-3-(trifluoromethyl)-4,5,6,7-tetrahydro-1 H-indazol-1-yl)-3-fluorophenyl)amino)cyclohexyl)oxy)-N,N,N-trimethyl-4-oxobutan-1-aminium chloride;
2-acetoxy-4-((5-((2-carba moyl-3-fluoro-5-(3,6,6-trimethyl-4-oxo-4, 5,6,7-tetrahydro-1 H-indazol-1 -yl)phenyl)amino)-2-methoxycyclohexyl)oxy)-N, N,N-trimethyl-4-oxobutan-1-aminium chloride;
2-acetoxy-4-((2-((6-carbamoyl-3-(3-ethyl-6,6-dimethyl-4-oxo-4,5,6, 7-tetrahydro-1H-indazol-1-yl)-2-fluorophenyl)amino)cyclohexyl)oxy)-N,N, N-trimethyl-4-oxobutan-1 -aminium chloride;
2-acetoxy-4-((4-((2-carbamoyl-5-(3-ethyl-6,6-d i methyl-4-oxo-4,5,6, 7-tetrahydro-1H-indazol-1 -yl)-3,4,6-trifluorophenyl)amino)cyclohexyl)oxy)-N, N,N-trimethyl-4-oxobutan-1 -aminium chloride;
2-acetoxy-4-((6-((2-carba moyl-3-fluoro-5-(3,6,6-trimethyl-4-oxo-4, 5,6,7-tetrahydro-1H-indazol-1-yl)phenyl)amino)-7-oxoazepan-3-yl)oxy)-N, N,N-trimethyl-4-oxobutan-1-aminium chloride;
2-acetoxy-4-((4-((2-carbamoyl-3-fluoro-5-(3,6,6-trimethyl-4-oxo-4, 5,6,7-tetrahydro-1H-indol-1-yl)phenyl)amino)tetrahydrofuran-2-yl)oxy)-N, N,N-trimethyl-4-oxobutan-1-aminium chloride;
2-acetoxy-4-((4-(2-carbamoyl-5-(3-(cyclopropylmethyl)-6, 6-dimethyl-4-oxo-4,5,6,7-tetrahydro-1H-indazol-1-yl)-4-fluorophenyl) amino)tetrahydrofuran-2-yl)oxy)-N,N,N-trimethyl-4-oxobutan-1-aminium chloride;
2-acetoxy-4-((4-((2-carbamoyl-3-fluoro-5-(3,6,6-trimethyl-4-oxo-4, 5,6,7-tetrahydro-1H-indazol-1-yl)phenyl)amino)cyclopent-2-en-1-yl)oxy)-N, N,N-trimethyl-4-oxobutan-1-aminium chloride;
2-acetoxy-4-(3-((2-carbamoyl-5-(3-ethyl-6,6-dimethyl-4-oxo-4,5,6, 7-tetrahydro-1H-indazol-1-yl)-3-fluorophenyl)amino)cyclobutoxy)-N,N, N-trimethyl-4-oxobutan-1-aminium chloride;
2-acetoxy-4-((4-((2-carbamoyl-3-fluoro-5-(3,6,6-trimethyl-4-oxo-4, 5,6,7-tetrahydro-1H-indazol-1-yl)phenyl)amino)tetrahydro-2H-pyran-2-yl) oxy)-N,N,N-trimethyl-4-oxobutan-1-aminium chloride;
2-acetoxy-4-((4-((6-carbamoyl-3-(3-ethyl-6,6-dimethyl-4-oxo-4,5,6, 7-tetrahydro-1H-indazol-1-yl)-2-fluorophenyl)amino)cyclohexyl)oxy)-N,N, N-trimethyl-4-oxobutan-1-aminium chloride;
2-acetoxy-4-((4((2-carbamoyl-3-fluoro-5-(3,6,6-trimethyl-4-oxo-4, 5,6,7-tetrahydro-1H-indazol-1-yl)phenyl)amino)tetrahydrofuran-2-yl)oxy)-N, N,N-trimethyl-4-oxobutan-1-aminium chloride;
2-acetoxy-4-((4-((6-carbamoyl-3-(3-(cyclopropylmethyl)-6, 6-dimethyl-4-oxo-4,5,6,7-tetrahydro-1H-indazol-1-yl)-2-fluorophenyl) amino)cyclohexyl)oxy)-N,N,N-trimethyl-4-oxobutan-1-aminium chloride;
2-acetoxy-4-(4-((2-carbamoyl-5-(3-ethyl-6,6-dimethyl-4-oxo-4,5,6, 7-tetrahydro-1H-indazol-1-yl)-3-fluorophenyl)amino)piperidin-1-yl)-N,N, N-trimethyl-4-oxobutan-1-aminium chloride;
2-acetoxy-4-((6-((2-carbamoyl-3-fluoro-5-(3,6,6-trimethyl-4-oxo-4, 5,6,7-tetrahydro-1H-indazol-1-yl)phenyl)amino)-7-oxoazepan-4-yl)oxy)-N,N,N-trimethyl-4-oxobutan-1-aminium chloride;
2-acetoxy-4-((4-((2-carbamoyl-3-fluoro-5-(3,6,6-trimethyl-4-oxo-4, 5,6,7-tetrahydro-1H-indazol-1-yl)phenyl)amino)cyclohexyl)oxy)-N,N, N-trimethyl-4-oxobutan-1-aminium chloride;
4-(6,6-dimethyl-4-oxo-3-(trifluoromethyl)-4,5,6,7-tetrahydro-1H-indazol-1-yl)-2-(piperidin-3-ylamino)benzamide;
2-acetoxy-4-(2-((2-carbamoyl-5-(3,6,6-trimethyl-4-oxo-4, 5,6,7-tetrahydro-1H-indol-1-yl)phenyl)amino)cyclopropoxy)-N,N,N-trimethyl-4-oxobutan-1-aminium chloride;
2-acetoxy-4-(((1r,4r)-4-((2-carbamoyl-5-(3,6,6-trimethyl-4-oxo-4, 5,6,7-tetrahydro-1H-indol-1-yl)phenyl)amino)cyclohexyl)oxy)-N,N,N-trimethyl-4-oxobutan-1-aminium chloride;
2-acetoxy-4-((1r,4r)-4-((2-carbamoyl-5-(2,6,6-trimethyl-4-oxo-3-(trifluoromethyl)-4, 5,6,7-tetrahydro-1H-indol-1-yl)phenylamino)cyclohexyloxy)-N,N,N-trimethyl-4-oxobutan-1-aminium chloride;
2-acetoxy-4-((1r,4r)-4-(2-carbamoyl-5-(3-ethyl-2,6,6-trimethyl-4-oxo-4, 5,6,7-tetrahydro-1H-indol-1-yl)phenylamino)cyclohexyloxy)-N,N, N-trimethyl-4-oxobutan-1-aminium chloride;
2-acetoxy-4-((1r,4r)-4-(2-carbamoyl-5-(6,6-dimethyl-4-oxo-3-(trifluoromethyl)-4, 5,6,7-tetrahydro-1H-indol-1-yl)phenylamino)cyclohexyloxy)-N,N,N-trimethyl-4-oxobutan-1-aminium chloride;
2-acetoxy-4-(((1r,4r)-4-((2-carbamoyl-5-(2,3,6,6-tetramethyl-4-oxo-4, 5,6,7-tetrahydro-1H-indol-1-yl)phenyl)amino)cyclohexyl)oxy)-N,N,N-trimethyl-4-oxobutan-1-aminium chloride;
2-acetoxy-4-(2-((1r,4r)-4-(2-carbamoyl-5-(3-ethyl-6,6-dimethyl-4-oxo-4, 5,6,7-tetrahydro-1H-indazol-1-yl)phenylamino)cyclohexyloxy)ethoxy)-N,N,N-trimethyl-4-oxobutan-1-aminium chloride;
2-acetoxy-4-((1 s,4s)-4-(allyl(2-carbamoyl-5-(6,6-dimethyl-4-oxo-3-(trifluoromethyl)-4, 5,6,7-tetrahydro-1H-indazol-1-yl)phenyl)amino)cyclohexyloxy)-N,N,N-trimethyl-4-oxobutan-1-aminium chloride; or
a pharmaceutically acceptable salt thereof.

22. A pharmaceutical composition comprising at least one compound or salt according to claim 1 and a pharmaceutically acceptable solvent, carrier, excipient, adjuvant or a combination thereof.

23. A method of treating cancer, inflammation, or arthritis comprising administering to a patient in need of such treatment a therapeutically effective amount of a compound or salt of claim 1.

24. (canceled)

25. A method of treating a disease or condition related to cell proliferation comprising administering a therapeutically effective amount of a compound or salt of claim 1 to a patient in need of such treatment.

26. (canceled)

27. A method for treating a subject suffering from a disease or disorder of proteins that are either client proteins for HSP-90 or indirectly affect its client proteins, comprising administering to a subject in need of such treatment a therapeutically effective amount of a compound or salt of claim 1.

28-31. (canceled)
